(19) United States
(12) Patent Application Publication
(10) Pub. No.: US 2025/0019750 A1
(43) Pub. Date: Jan. 16, 2025

(54) ENZYME-FREE ISOTHERMAL EXPONENTIAL AMPLIFICATION

(71) Applicants: President and Fellows of Harvard College, Cambridge, MA (US); Dana-Farber Cancer Institute, Inc., Boston, MA (US)

(72) Inventors: William M. Shih, Cambridge, MA (US); Dionis Minev, Cambridge, MA (US); Anastasia Ershova, Cambridge, MA (US)

(73) Assignees: President and Fellows of Harvard College, Cambridge, MA (US); Dana-Farber Cancer Institute, Inc., Boston, MA (US)

(21) Appl. No.: 18/710,722

(22) PCT Filed: Nov. 18, 2022
(86) PCT No.: PCT/US2022/050352

§ 371 Date: May 16, 2024

Related U.S. Application Data
(60) Provisional application No. 63/281,377, filed on Nov. 19, 2021.

(51) Int. Cl. C12Q 1/6844

(52) U.S. Cl. CPC C12Q 1/6844

(57) ABSTRACT

Provided herein, in some embodiments, are methods, compositions and kits for controlling nucleation and assembly of molecular nanostructures, microstructures and macrostructures.

### RELATED APPLICATION

This application claims the benefit under 35 U.S.C. § 119 (e) of U.S. provisional application No. 63/281,377, filed Nov. 19, 2021, which is incorporated by reference herein in its entirety.

### FEDERALLY SPONSORED RESEARCH

This invention was made with government support under N00014-18-1-2566 awarded by the Department of Defense/Office of Naval Research. The government has certain rights in the invention.

### BACKGROUND

Infectious disease is one of the leading causes of preventable death in the world and accounts for 50% of the top 10 causes of death in low-income countries. These deaths can be prevented by providing a means for conducting frequent testing. Accordingly, rapid and affordable diagnostics have the potential of significantly reducing the global burden of infectious disease. Furthermore, such screening approaches could also aid in early cancer diagnosis, which has been shown to significantly improve survival prospects in most cancer types. Current diagnostics methods like PCR, LAMP and CRISPR-based strategies all require enzymes, and often also require complex equipment (e.g., thermocyclers), increasing their cost and reducing accessibility. This presents a significant bottleneck in the development of highly sensitive and robust diagnostics that are affordable and amenable to low-resource settings or at-home testing. Thus, novel diagnostic methods having increased speed and decreased cost are needed.

### SUMMARY

Provided herein, in some embodiments, is a technology (including, for example, methods, compositions and kits) for controlling nucleation and hierarchical assembly (programmable self-assembly) of molecular structures, such as nucleic acid (e.g., DNA) and/or protein nanostructures. This technology, referred to herein as ‘crisscross cooperative assembly’ can be used to program and rapidly assemble structures that only originate from provided macromolecular ‘seeds’ and/or in response to a change in local environment, thus may be considered a ‘zero-background’ assembly method. Further, such methods provide, in some embodiments, enzyme-free and linear amplification. In some embodiments, such methods utilize isothermal exponential and branching amplification. The system described herein imposes an intrinsically high energetic barrier against spontaneous nucleation of structures, even in the presence of high concentrations of each individual component. This is achieved, in part, through the design of cooperative binding sites on individual biomolecular subunits that require simultaneous engagement with a large number of other subunits to achieve stable attachment.

Nucleation, in some embodiments, is triggered by providing a biomolecule ‘seed’ (or biomolecule comprising seed strands) that resembles a pre-existing structural interface (presents multiple weak binding sites for stable capture of the next subunit). Addition of a seed (or biomolecule comprising seed strands) that can stably capture individual subunits effectively bypasses the activation energy barrier against spontaneous nucleation to drive higher-order assembly of a microscale structure. In other embodiments, nucleation is triggered by a change in the local environment (e.g., a change in pH and/or temperature). Components can be continually added to the structures such that their growth in one-dimension, two-dimensions or three-dimensions is potentially as large as for other polymerization or crystallization processes. Non-linear crisscross cooperative assembly, in some embodiments, involves the use of self-assembling nucleic acids comprising single-stranded extensions that form a crisscross ribbon and complementary slat strands (also referred to as ‘growth-slats’), which allow for the scission of the ribbon at defined locations to produce fragments with additional growth fronts and/or initiation of the polymerization of a branch from the same core ribbon.

Accordingly, in some aspects, the disclosure provides a method comprising incubating in a reaction mixture: (a) a set of nucleic acid strands that bind to each other to form a crisscross ribbon comprising a first layer of strands and a second layer of strands, wherein the strands of the first layer are nonparallel to and bound to the strands of the second layer, (b) a biomolecule comprising seed strands to which strands of the first layer and/or second layer of the crisscross slat are bound; and (c) slat strands, each of which binds to multiple strands of the crisscross ribbon that are bound to the seed strands, thereby displacing the seed strands from the crisscross slat. In some embodiments, the biomolecule is a protein biomolecule, nucleic acid biomolecule, organic small molecule, or saccharide. In some embodiments, the nucleic acid biomolecule is a DNA nanostructure (e.g., DNA origami).

In some aspects, the disclosure provides a method comprising incubating in a reaction mixture: (a) a set of nucleic acid strands that bind to each other to form a crisscross ribbon comprising a first layer of strands and a second layer of strands, wherein the strands of the first layer are nonparallel to and bound to the strands of the second layer, and (b) slat strands, each of which binds to multiple strands of the first layer of strands that are bound to the strands of the second layer, thereby displacing the strands of the first layer from the strands of the second layer.

In some embodiments, the first layer of strands of the crisscross slat comprises parallel strands, and the second layer of strands of the crisscross slat comprises parallel strands that are perpendicular to and bound through cooperative binding sites to the strands of the first layer. In some embodiments, each of the cooperative binding sites forms a helical half-turn. In some embodiments, a full helical turn of DNA comprises or consists of ten nucleotides in length. Thus, in some embodiments, a helical half-turn comprises or consists of 5 or 6 nucleotides. In some embodiments, each of the cooperative binding sites comprises 3-10 nucleotide base pairs, optionally wherein each of the cooperative binding sites comprises 5-6 nucleotide base pairs.

In some embodiments, the biomolecule comprises (i) a first subset of seed strands to which a first subset of strands of the first layer of the crisscross slat are bound, and/or (ii) a second subset of seed strands to which a second subset of strands of the second layer of the crisscross slat are bound. In some embodiments, slat strands comprise (i) a first subset of strands, each of which binds to multiple strands of the first layer of the crisscross slat and (ii) a second subset of strands, each of which binds to multiple strands of the second layer of the crisscross slat, wherein binding of the slat strands to the strands of the crisscross slat displaces the crisscross slat from the seed strands, thereby displacing the crisscross slat from the biomolecule.

In some embodiments, each nucleic acid strand of the first layer of strands comprises a single-stranded extension at one or both of its terminal ends. In some embodiments, each nucleic acid strand of the second layer of strands comprises a single-stranded extension at one or both of its terminal ends.

In some embodiments, there are 2, 3, 4, 5, 6, 7, 8, 9 or 10 cooperative binding sites.

In some embodiments, the nucleotides of the first layer of strands are complementary to the nucleotides of the second layer of strands. In some embodiments, the nucleotides of first layer of strands comprise at least one wobble base-pairing, mismatched base-pairing, or deletion relative to the nucleotides of second layer of strands that bind to the first layer.

In some embodiments, the method is performed at a temperature between 20-60° C., optionally 46-52° C.

In some embodiments, the seed strands are displaced from the crisscross ribbon by (i) tochold-mediated strand displacement; (ii) inclusion of gamma cut slats; inclusion of an engineered restriction site in a slat strand; (iii) inclusion of an unnatural or modified base in a strand of the crisscross ribbon and/or a slat strand; (iv) inclusion of small molecules that can function to accelerate scission in the reaction mixture; (v) inclusion of pH responsive elements in a strand of the crisscross ribbon and/or a slat strand; (vi) inclusion of a crosslinking or ligation junction in a strand of the crisscross ribbon and/or a slat strand; (vii) mechanical rupturing of crisscross ribbons; (viii) inclusion of photothermal elements in a strand of the crisscross ribbon and/or a slat strand; (ix) polymerase-based scission; (x) inclusion of thermoactivated slat strands; (xi) inclusion of nicking sites in a strand of the crisscross ribbon and/or a slat strand; and/or (xii) inclusion of exonucleases in the reaction mixture.

Some aspects of the disclosure provide a method of detection of a biomarker, comprising: combining in a reaction mixture (a) a set of nucleic acid strands that bind to each other to form a crisscross ribbon comprising a first layer of strands and a second layer of strands, wherein the strands of the first layer are nonparallel to and bound to the strands of the second layer, (b) a biomolecule comprising seed strands to which strands of the first layer and/or second layer of the crisscross slat are bound, wherein the biomolecule seed is representative of the biomarker; and (c) slat strands, each of which binds to multiple strands of the crisscross ribbon that are bound to the seed strands; and incubating the reaction mixture under conditions that result in production of a branched nucleic acid nanostructure, wherein visualization of the nanostructure enables detection of the biomarker.

In some embodiments, the biomolecule comprises a biomarker binding partner that specifically binds to the biomarker.

Some aspects of the disclosure provide a method of detection of a biomarker, comprising: combining in a reaction mixture (a) a sample comprising a biomarker; and (b) a nucleic acid nanostructure comprising (i) a nucleic acid scaffold strand and nucleic acid staple strands capable of assembling into multiple stacked parallel loops, and (ii) a crisscross ribbon comprising a first layer of strands and a second layer of strands, wherein the strands of the first layer are nonparallel to and bound to the strands of the second layer, and slat strands, wherein each of the slat strands binds to multiple strands of the first layer of strands that are bound to the strands of the second layer, wherein the crisscross ribbon binds to the loops of (i); wherein a biomarker binding partner that specifically binds to the biomarker is linked to each of the crisscross ribbons such that in the presence of the biomarker the biomarker binding partners bind to the biomarker and the nucleic acid nanostructure folds into multiple stacked parallel loops; and incubating the reaction mixture to assemble multiple stacked parallel loops.

In some embodiments, the biomolecule seed is attached to the biomarker, optionally wherein the biomolecule seed is attached to the biomarker via an affinity agent.

In some embodiments, the biomolecule seed is a segment of the biomarker, wherein the biomarker comprises a nucleic acid.

In some embodiments, the method further comprises imaging the nanostructure.

In some embodiments, each nucleic acid strand of the first layer of strands comprises a single-stranded extension at one or both of its terminal ends. In some embodiments, each nucleic acid strand of the second layer of strands comprises a single-stranded extension at one or both of its terminal ends.

In some embodiments, the first layer of strands of the crisscross slat comprises parallel strands, and the second layer of strands of the crisscross slat comprises parallel strands that are perpendicular to and bound through cooperative binding sites to the strands of the first layer. In some embodiments, each of the cooperative binding sites forms a helical half-turn. In some embodiments, each of the cooperative binding sites comprises 3-10 nucleotide base pairs, optionally wherein each of the cooperative binding sites comprises 5-6 nucleotide base pairs.

In some embodiments, the biomolecule comprises (i) a first subset of seed strands to which a first subset of strands of the first layer of the crisscross slat are bound, and/or (ii) a second subset of seed strands to which a second subset of strands of the second layer of the crisscross slat are bound.

In some embodiments, the slat strands comprise (i) a first subset of strands, each of which binds to multiple strands of the first layer of the crisscross slat and (ii) a second subset of strands, each of which binds to multiple strands of the second layer of the crisscross slat, wherein binding of the slat strands to the strands of the crisscross slat displaces the crisscross slat from the seed strands, thereby displacing the crisscross slat from the biomolecule.

In some embodiments, the nucleotides of the first layer of strands are complementary to the nucleotides of the second layer of strands. In some embodiments, the nucleotides of first layer of strands comprise at least one wobble base-pairing, mismatched base-pairing, or deletion relative to the nucleotides of second layer of strands that bind to the first layer.

In some embodiments, the method is performed at a temperature between 20-60° C., optionally 46-52° C. In some embodiments, the biomarker binding partner that specifically binds to the biomarker is an antibody or aptamer.

Further aspects of the disclosure provide a method of detection of an environmental change in a sample, comprising: combining in the sample (a) a set of nucleic acid strands that bind to each other to form a crisscross ribbon comprising a first layer of strands and a second layer of strands, wherein the strands of the first layer are nonparallel to and bound to the strands of the second layer, and (b) slat strands, each of which binds to multiple strands of the first layer of strands that are bound to the strands of the second layer following an environmental change, thereby displacing the strands of the first layer from the strands of the second layer.

In some embodiments, the environmental change is a pH change, a temperature change, or a change in the concentration of one or more metal ions.

### BRIEF DESCRIPTION OF THE DRAWINGS

FIGS. 1A-1E provide an overview of exponential amplification strategy via ribbon scission. FIG. 1A shows an abstraction of linear ribbon assembly. FIG. 1B shows an exemplary scission strategy in which cutting of linear ribbon between repeating units leads to exponential amplification of growing fronts, as every repeating unit is capable of seeding further linear growth and subsequent scission. FIG. 1C shows a detailed depiction of a first stage of a cutting mechanism in which nucleic acids have single-stranded extensions to which cut-slats can bind. FIG. 1D shows a detailed depiction of a second stage of a cutting mechanism in which tochold-mediated strand displacement leads to generation of two distinct ribbon fragments that are both competent for subsequent growth. FIG. 1E shows a detailed depiction of a third stage of a cutting mechanism in which tochold regions to which cut-slats bind to the single-stranded extensions to cause cutting. Cut-slats can be varied in length (e.g., 2, 4, or 6 segments as in the insets). The highlighted regions can be programmed to contain wobble base-pairs or deletions to increase the rate of cutting.

FIGS. 2A-2B provides a transmission electron microscopy (TEM) characterization of exponential amplification (FIG. 2A) and a corresponding abstraction of the experimental design (FIG. 2B). FIG. 2A shows linear ribbon assembly (only growth-slats) (left panel) and crisscross ribbon assembly with both cut-slats and growth-slats, resulting in a larger number of short fragments (right panel). Scale bars are 500 nm.

FIGS. 3A-3E provide an agarose gel electrophoresis (AGE) characterization of exponential amplification. FIG. 3A shows that exponential assembly of a crisscross ribbon is detectable in the presence of an initiating DNA origami seed (+S; center lane), but not in the absence of an initiating DNA origami seed (−S; right lane). The left lane is a control experiment containing DNA origami seed in the absence of self-assembling nucleic acids. FIG. 3B shows that longer tocholds result in a greater degree of cutting in the presence of an initiating DNA origami seed. FIG. 3C shows that deletions or wobble base pairs bias assembly towards more cutting in the presence of an initiating DNA origami seed. FIG. 3D shows the temperature robustness of exponential assembly. There is no spurious nucleation detectable across a range of temperatures for exponential assembly (‘cutting’), and significantly more amplification is seen compared to linear growth conditions (‘linear’). FIG. 3E shows an abstraction of the experimental design of the exponential assembly used to generate the data presented in FIG. 3A. FIG. 3F shows an abstraction of the experimental design of the exponential assembly with long tocholds used to generate the data presented in FIG. 3B. FIG. 3G shows an abstraction of the experimental design of the exponential assembly with medium tocholds used to generate the data presented in FIG. 3B. FIG. 3H shows an abstraction of the experimental design of the exponential assembly with short tocholds used to generate the data presented in FIG. 3B. FIG. 3I shows an abstraction of the experimental design of the exponential assembly used to generate the data presented in FIG. 3D.

FIGS. 4A-4E provides an overview of an amplification strategy using dendrimeric growth. FIG. 4A shows an abstraction of dendrimeric growth with branches on both sides of a crisscross ribbon. FIG. 4B shows an example implementation of dendrimeric growth using DNA strands with branches on both sides of a crisscross ribbon. FIG. 4C shows an abstraction of dendrimeric growth with branches on only one side of the ribbon. FIG. 4D shows an example implementation of dendrimeric growth using DNA strands with branches on only one side of the ribbon. FIG. 4E shows a TEM micrograph of two dendrimeric ribbons. Scale bar is 100 nm.

### DETAILED DESCRIPTION

Nature achieves rapid and nucleation-limited growth of cytoskeletal filaments such as actin and microtubules. This is achieved by securing each additional subunit by weak interactions to 2-3 already attached subunits at the growing end of the filament. This means that if any two monomers bind to each other in solution, they will rapidly (e.g., within milliseconds) dissociate from each other, because the single interaction is so weak. It is only after four subunits come together simultaneously—a rare event—that a stable nucleus will be formed. Therefore, untriggered spontaneous nucleation will be rare. Conversely, nucleation can be triggered by providing a biomolecule “seed” (or biomolecule comprising seed strands) that mimics a fully formed filament end and/or by changing the local environment of the nucleic acids (e.g., pH and/or temperature).

Rapid and nucleation-limited growth are very useful features for programmable self-assembly, however technological modification of natural filaments such as actin or microtubules has many current drawbacks: (1) there is a limited understanding of how to tune the interaction strength between subunits; (2) the level of cooperativity is relatively low (the weak interactions upon binding are spread only over 2-3 subunits), therefore the suppression of spontaneous nucleation is not as robust as it could be; and (3) growth is limited to one-dimension (filament formation).

Rapid, reversible, zero-background, triggered nucleation and growth, as provided herein, can have useful applications in nanotechnology and biotechnology, such as ultrasensitive detection, and templates for miniaturized materials. Such approaches to triggered nucleation and growth, including crisscross polymerization, represent a novel and surprisingly effective approach of DNA self-assembly that allows for the formation of several micron-long nucleic acid structures while maintaining absolute control over their nucleation.

For example, single-stranded DNA (ssDNA) “slats” weave over and under six or more previously captured slats, forming weak yet specific half-turn interactions (5-6 nucleotides (nt)) with each one of these slats to form crisscross ribbon structures. Ribbon growth propagates by linear addition of perpendicular slats as new binding sites are made available by every subsequent addition. Such interactions with more than just nearest-neighbor slats allow for extremely high levels of cooperativity, meaning that rapid growth can be attained at conditions with virtually no spontaneous nucleation, as any spuriously interacting slats do not have sufficient binding energy to initiate stable ribbon formation. However, the addition of a seed that pre-organizes the initial set of binding sites is one mechanism for allowing the system to bypass this large entropic barrier and thereby leads to rapid ribbon assembly. In the context of diagnostics, this seed can be formed from a biomarker (e.g., by directly incorporating a nucleic acid biomarker, or by a combination of affinity agents and kinetic proofreading for protein biomarkers).

As used herein, a “crisscross ribbon” is a nucleic acid nanostructure comprising nucleic acid (e.g., DNA and/or RNA) strands that may comprise two or more layers of nucleic acids that are non-parallel (e.g., perpendicular) to one another. In some embodiments, a crisscross ribbon comprises a first layer of nucleic acid strands (e.g., ssDNA strands) that are non-parallel (e.g., perpendicular) to a second layer of nucleic acid strands (e.g., ssDNA strands).

### Crisscross Cooperative Assembly

The crisscross cooperative assembly technology as provided herein is based on a concept that may apply to many self-assembling molecules, including nucleic acids and proteins. For simplicity and ease of understanding, however, reference herein primarily will address crisscross cooperative assembly in the context of nucleic acids, such as deoxyribonucleic acid (DNA). A crisscross cooperative assembly system, in some embodiments, uses three basic components: a seed molecule (e.g., a biomolecule comprising seed strands or a seed formed from a biomarker), a set of nucleic acid strands that bind to each other to form a crisscross ribbon comprising a first layer of strands and a second layer of strands, and slat strands that bind to one or both of the layers of the crisscross ribbon.

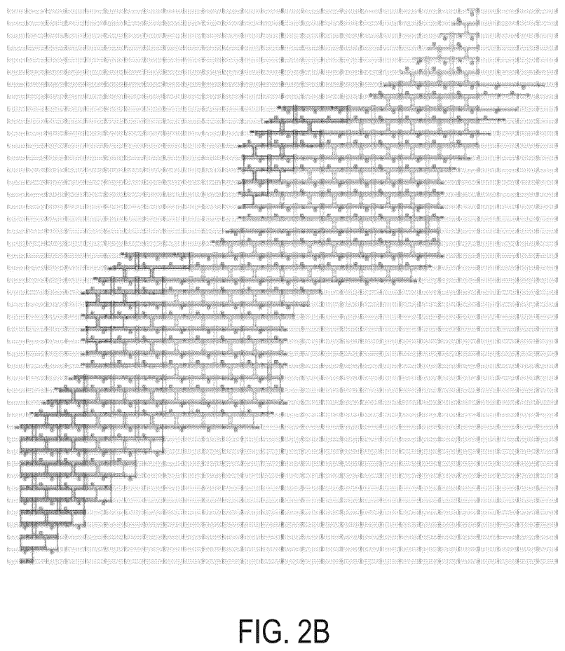

An example of a crisscross cooperative assembly is provided in Minev et. al., Nat Communications. 2021 Mar. 19; 12 (1): 1741. doi: 10.1038/s41467-021-21755-7., wherein a crisscross ribbon is formed by assembling a crisscross ribbon in the presence of a nucleating structure (e.g., a ‘seed’). See, e.g., FIG. 2 of Minev et. al. The final structure, in this example, includes layers of aligned molecular rods, where each layer is rotated by some amount (e.g., 90 degrees) relative to the layer below and above. For example, one layer may be perpendicular to another adjacent (directly above or below) layer. In some embodiments, one layer is rotated 20, 30, 40, 50, 60, 70, 80 or 90 degrees relative to an adjacent layer (measured alone the length of nucleic acid of the first layer, for example). Each intersection between nucleic acids on adjacent layers adds a small binding energy; any given nucleic acids intersects with a large number of nucleic acids below and above, and the net binding energy can be tuned (e.g., by adjusting the design of the binding interface, for example, the number of base pairs, or by adjusting subunit concentration, temperature, or salt concentration) to be large enough to achieve stable (irreversible) or slightly favorable (reversible) attachment as desired. Before assembly initiates, any spontaneous crossing between two nucleic acids in solution is short-lived, as the net energy is very low because there is only one interaction. Thus, a nucleic acid can be stably (or else slightly favorably (reversibly)) added to a pre-existing crisscross structure (many attachment points can immediately be realized), but a structure will not spontaneously assemble in the absence of a pre-existing one. There should be no growth unless a structural mimic of a pre-existing crisscross structure—a seed—is added to the solution.

Example protocols for crisscross assembly can also be found in Minev, D.; Wintersinger, C. M.; Ershova, A.; Shih, W. M. Robust Nucleation Control via Crisscross Polymerization of DNA Slats. bioRxiv 2019, 2019.12.11.873349.; and Minev, D.; Wintersinger, C.; Shih, W. M. Crisscross Cooperative Self-Assembly. International Patent Publication WO 2018026880, published Feb. 8, 2018; the entire contents of each of which are incorporated herein by reference in their entireties.

A biomolecule comprising seed strands may be a protein biomolecule, nucleic acid biomolecule, organic small molecule, or saccharide (e.g., polysaccharide). A protein biomolecule may be a protein or peptide of any length or origin. In some embodiments, a protein biomolecule is an antibody (e.g., an antibody that specifically binds to a target protein). In some embodiments, a nucleic acid biomolecule may be a DNA or RNA molecule. In some embodiments, a nucleic acid biomolecule comprises RNA and DNA. In some embodiments, a nucleic acid biomolecule is a DNA nanostructure (e.g., DNA origami). In some embodiments, seed strands may be covalently or non-covalently attached to the biomolecule. For example, in some embodiments, a protein biomolecule is covalently attached to seed strands using conventional techniques (e.g., crosslinking techniques). In some embodiments, seed strands are attached to the biomolecule via a linker. A linker may be a tricthylene glycol spacer (e.g., iSp9 or Spacer 18), a nucleic acid linker, or an affinity agent (e.g., biotin and/or streptavidin). A biomolecule may comprise a biomarker binding partner (e.g., an antibody, aptamer, or protein binding fragment). In some embodiments, a biomarker binding partner specifically binds to a biomarker (e.g., a biomarker for detection).

A layer of strands in a crisscross ribbon may be perpendicular and/or nonparallel to another adjacent (directly above or below) layer. In some embodiments, one layer is perpendicular and nonparallel to another adjacent layer. In some embodiments, one layer is nonparallel to another adjacent layer. In some embodiments, a first layer of strands is nonparallel to a second layer (e.g., an adjacent layer) when the first layer is rotated by 20, 25, 30, 35, 40, 45, 50, 55, 60, 65, 70, 75, 80, 85 or 90 degrees relative to the second layer. In some embodiments, a first layer of strands is nonparallel to a second layer (e.g., an adjacent layer) when the first layer is rotated by 20-90, 25-90, 30-90, 35-90, 60-90, 20-45, 25-50, 30-55, 35-60, 40-65, 45-70, 40-75, 50-80, 55-85 or 75-90 degrees relative to the second layer.

Nanostructures or layers of nucleic acid strands bind to each other through cooperative binding sites. A “cooperative binding site” may be the location at which two nanostructures interact (hybridize/bind) or two layers of nucleic acids strands (e.g., two layers of nucleic acid strands that form a crisscross ribbon). For example, a nucleating nanostructure may be programmed with multiple nucleotide base sequences, each of which is complementary to a nucleotide base sequence of an additional nanostructure

Cooperative binding sites may also be used to assemble nucleic acid (e.g., DNA) slats onto another nucleic acid scaffold structure in a similar manner. For example, DNA slats may be appended to a nucleic acid scaffold (queen or seed) to secure the two- or three-dimensional shape of the scaffold structure. In the example, DNA slats are used to secure (hold together) the barrel shape of a larger scaffold nanostructure. “Growth” of these slats along the scaffold through cooperative binding sites results in a barrel-like shape that may be visualized by microscopy, for example.

Cooperative binding sites (e.g., plug and socket sequences) are arranged on a nucleating nanostructure or crisscross ribbon in a spatial configuration that facilitates binding and alignment of the initial e.g., scaffold) nanostructures. The length of a cooperative binding site may vary, depending in part on the desired strength (e.g., strong v. weak) of the intended interaction between two molecules having complementary sites. In some embodiments, a cooperative binding site has a length of 5-50 nucleotides. For example, a cooperative binding site may have a length of 5-40, 5-30, 5-20, 5-10, 5-15, 10-50, 10-40, 10-30, 10-20, 30-50, 30-40, or 40-50 nucleotides. In some embodiments, a cooperative binding site has a length of 5, 10, 15, 20, 25, 30, 35, 40, 45, or 50 nucleotides. A single plug strand and/or socket strand may have a length of 5-20 (e.g., 5, 6, 7, 8, 9, 10, 11, 12, 13, 14, 15, 16, 17, 18, 19 or 20) nucleotides, for example. In some embodiments, a cooperative binding site may have a length of 4, 5, 6, 7, 8, 9 or 10 nucleotides.

The number of cooperative binding sites on a nanostructure or crisscross ribbon or tochold domain may also vary. In some embodiments, the number of cooperative binding sites is 2-1000. For example, the number of cooperative binding sites may be 2-900, 2-800, 2-700, 2-600, 2-500, 2-400, 2-300, 2-200, or 2-100. In some embodiments, the number of cooperative binding sites is 2-10, 2-15, 2-20, 2-25, 2-30, 2-35, 2-40, 2-45 or 2-50. In some embodiments, the number of cooperative binding sites is 2-15, 2-20, 2-25, 2-30, 2-35, 2-40, 2-45 or 2-50. In some embodiments, the number of cooperative binding sites is 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100. In some embodiments, a short tochold domain comprises 2 cooperative binding sites. In some embodiments, a medium tochold domain comprises 3 cooperative binding sites. In some embodiments, a long tochold domain comprises 4 cooperative binding sites.

The distance between cooperative binding sites may also vary. In some embodiments, the distance between two cooperative binding sites on the same nanostructure is 20-1000 angstroms. For example, the distance between two cooperative binding sites on a nanostructures may be 20-900, 20-800, 20-700, 20-600, 20-500, 20-400, 20-300, 20-200, 20-100, 50-1000, 50-900, 50-800, 50-700, 50-600, 50-500, 50-400, 50-300, 50-200, or 50-100 angstroms. In some embodiments, the distance between two cooperative binding sites on a nanostructures is 20, 25, 30, 35, 40, 45, 50, 55, 60, 65, 70, 75, 80, 85, 90, 95, 100, 125, 150, 175, 200, 250, 300, 350, 400, 450 or 500 angstroms.

In some embodiments, the distance between cooperative binding sites, for example, the distance between plug strands (and/or between socket strands) may be 5 to 100 nucleotides (or nucleotide base pairs (bp)). In some embodiments, the distance between plug strands (and/or between socket strands) is 5-20, 5-25, 5-50 or 5-100 nucleotides. In some embodiments, the distance between plug strands (and/or between socket strands) is 5, 10, 15, 20, 25, 30, 35, 40, 45, 50, 55, 60, 65, 70, 75, 80, 85, 90, 95 or 100 nucleotides. In some embodiments, the distance between plug strands (and/or between socket strands) is 5, 6, 7, 8, 9, 10, 11, 12, 13, 14, 15, 16, 17, 18, 19, 20 or 21 nucleotides. In some embodiments, the distance between plug strands (and/or between socket strands) is 42+/−21 nucleotides. For example, the distance between plug strands (and/or between socket strands) may be 21, 42 or 63 nucleotides. In some embodiments, the distance between plug strands (and/or between socket strands) is 42 nucleotides.

One nucleotide unit measures 0.33 nm. Thus, in some embodiments, the distance between cooperative binding sites, for example, the distance between plug strands (and/or between socket strands) may be 5 to 35 nanometers (nm). In some embodiments, the distance between plug strands (and/or between socket strands) is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nm. In some embodiments, the distance between plug strands (and/or between socket strands) is 14+/−7 nm. For example, the distance between plug strands (and/or between socket strands) may be 7, 14 or 21 nm. In some embodiments, the distance between plug strands (and/or between socket strands) is 14 nucleotides.

In some embodiments, the distance between two cooperative binding sites on a nanostructure is evenly spaced, while in other embodiments, the distances may vary. For example, the distance between a first cooperative binding site and a second cooperative binding site may be 30 angstroms, while the distance between the second cooperative binding site and a third may be 30 angstroms, 40 angstroms or 50 angstroms.

Two or more nanostructures are considered “aligned” if they are oriented in the same direction relative to one another. For example, the 5′ ends (or 3′ ends) of the nanostructures maybe facing the same direction along its y axis.

A nucleating nanostructure or seed can be used to initiate assembly of the first (initial) and second (and, thus, subsequent, e.g., third, fourth, fifth, etc.) subsets of nanostructures or layers of a crisscross ribbon, and binding of the first subset is required to initiate assembly of the second subset. A “nucleating nanostructure” is any nanostructure or crisscross ribbon programmed with binding sites that interacts strongly (e.g., irreversibly) with binding sites on an additional nanostructure and aligns them for recruitment of subsequent nanostructures. That is, the binding sites between a nucleating nanostructure and one or more additional nanostructures should be strong enough that the one or more additional nanostructures bind to and align along the nucleating nanostructures and do not dissociate from the nucleating nanostructure under reaction conditions (e.g., isothermal, physiological conditions). A nucleating nanostructure may have a two-dimensional or a three-dimensional shape, for example.

Additional subsets of nanostructures may be added to the crisscross cooperative assembly system to propagate growth of the end nanostructure. For example, third, fourth and fifth subsets of nanostructures may be added. Binding of the nanostructures of the second subset to the first subset is required to initiate assembly of the nanostructures of the third subset; binding of the nanostructures of the third subset to the second subset is required to initiate assembly of the nanostructures of the fourth subset; and so on. The user-defined end structure may be assembled in one dimension, two dimensions or three-dimensions.

Each subset of nanostructures should follow a specific set of binding energy parameters. More specifically, an initial subset of nanostructures should bind strongly (e.g., irreversibly) to and form an aligned layer (where each nanostructure is oriented in the same direction relative to one another) along the nucleating nanostructure or crisscross ribbon. The nanostructures of an initial subset should not interact with (bind to) each other. Likewise, nanostructures of a subsequent subset should not interact with (bind to) each other. Further, in the absence of a nucleating structure, any nanostructure from an initial subset should have only one weak (reversible) interaction with any other nanostructure (e.g., nanorod or nucleic acid layers) from a subsequent subset. In the presence of a nucleating structure, a single nanostructure from an initial subset may interact with more than one nanostructure) from a subsequent subset, and a single nanostructure from a subsequent subset may interact with more than one nanostructure from an initial subset.

A “strong interaction” refers to binding that is engaged more than 50% (e.g., more than 60%, 70%, 80% or 90%) of the time that the binding nucleic acids are in a reaction together (the dissociation constant is lower than the concentration of the species in excess).

A “weak interaction”-refers to binding that is engaged less than 1% of the time that the binding nucleic acids are in a reaction together (the dissociation constant is at least 100 times higher than the concentration of the species in excess).

A nucleating nanostructure may bind to two or more other nanostructures. In some embodiments, a nucleating nanostructure binds to 5-1000 nanostructures. For example, a nucleating nanostructure may bind to 3-900, 3-800, 3-700, 3-600, 3-500, 3-400, 3-300, 3-200, or 3-100 nanostructures. In some embodiments, a nucleating nanostructure binds to 3-10, 3-15, 3-20, 3-25, 3-30, 3-35, 3-40, 3-45 or 3-50 nanostructures. In some embodiments, a nucleating nanostructure binds to 10-15, 10-20, 10-25, 10-30, 10-35, 10-40, 10-45 or 10-50 nanostructures. In some embodiments, a nucleating nanostructure binds to 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nanostructures.

Thus, a single subset of nanostructures (nanostructures programmed to interact with a single nucleating nanostructure) may comprise 3-900, 3-800, 3-700, 3-600, 3-500, 3-400, 3-300, 3-200, or 3-100 nanostructures. In some embodiments, a single subset of nanostructures comprises 3-10, 3-15, 3-20, 3-25, 3-30, 3-35, 3-40, 3-45 or 3-50 nanostructures. In some embodiments, a single subset of nanostructures comprises 10-15, 10-20, 10-25, 10-30, 10-35, 10-40, 10-45 or 10-50 nanostructures. In some embodiments, a single subset of nanostructures comprises 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nanostructures (e.g., DNA nanorods).

A “subset of nanostructures” refers to a specific group of nanostructures that are similar in size (have similar dimensions) and structure/shape and are programmed to bind to either the nucleating nanostructure (the initial subset) or to a pre-existing layer formed by alignment and binding of other nanostructures that have already aligned and bound to the nucleating structure or nanostructures of another pre-existing layer.

Nanostructures within a defined subset are programmed not bind to each other. Thus, in some embodiments, less than 10% of the nanostructures of a subset bind to another nanostructure of the same subset. In some embodiments, less than 9%, less than 8%, less than 7%, less than 6%, less than 5%, less than 4%, less than 3%, less than 2%, less than 1%, less than 0.5%, less than 0.2%, or less than 0.1% of the nanostructures of a subset bind to another nanostructure of the same subset. In some embodiments, none of the nanostructures of a subset bind to another nanostructure of the same subset.

With crisscross cooperative assembly, nanostructures are aligned to form multiple layers, each layer rotated by some degree relative to adjacent layers (above and below). The top layer of aligned layers is rotated nonparallel (e.g., 90 degrees) relative to the bottom layer of aligned layers. The degree of rotation between two adjacent layers may vary. In some embodiments, one layer is rotated 10-90 degrees, 20-90 degrees, 30-90 degrees, 40-90 degrees, 50-90 degrees, 60-90 degrees, 70-90 degrees, or 80-90 degrees relative to an adjacent layer. In some embodiments, one layer is rotated 10, 15, 20, 25, 30, 35, 40, 45, 50, 55, 60, 65, 70, 75, 80, 85, or 90 degrees relative to an adjacent layer.

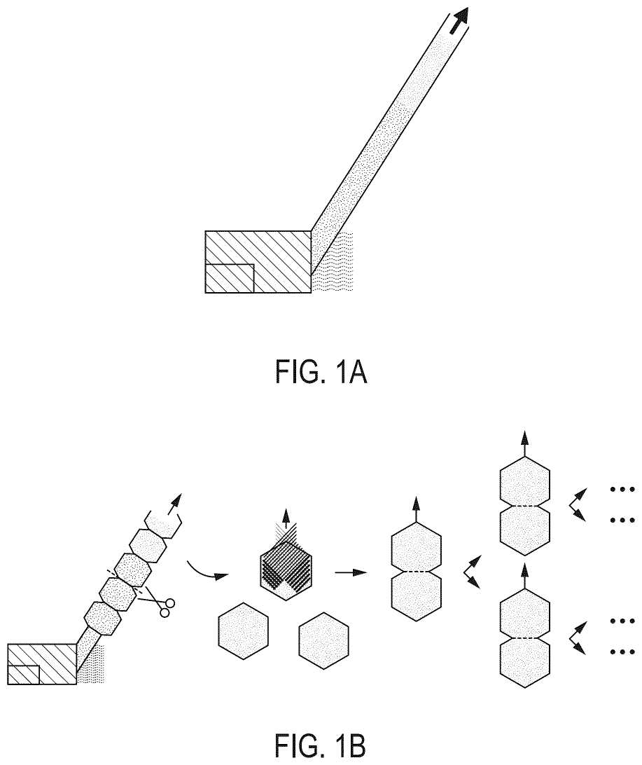

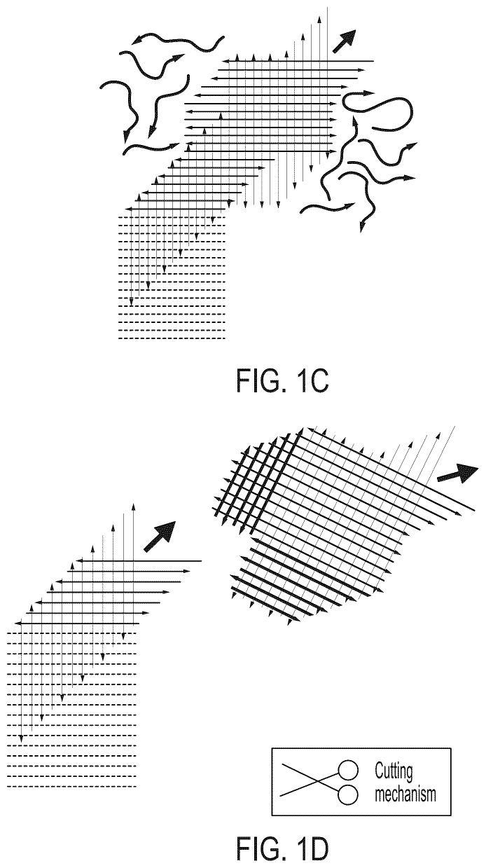

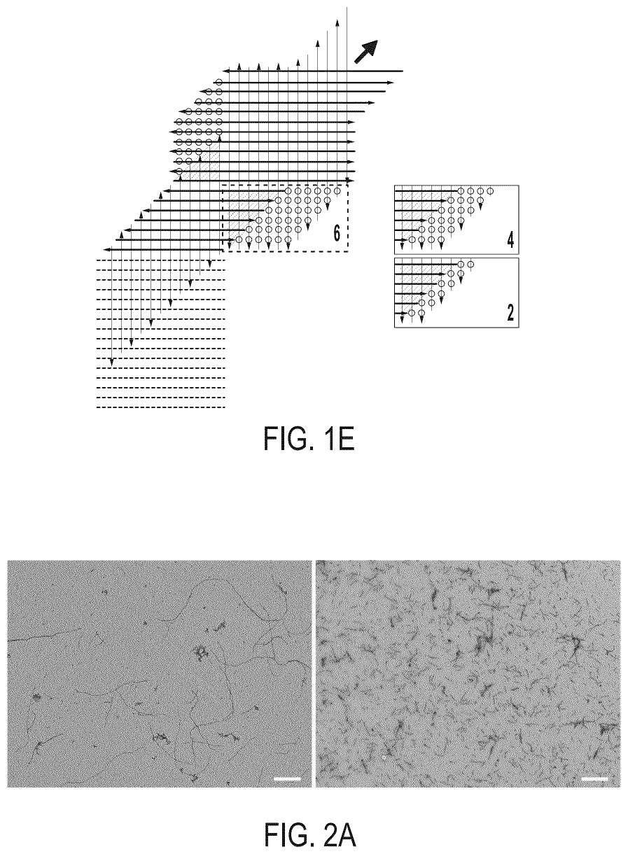

In some embodiments, crisscross cooperative assembly comprises exponential amplification via ribbon scission. For example, as a crisscross ribbon assembles, the sequential binding of x-slats and y-slats (e.g., perpendicular layers of nucleic acids) generates a concerted arrangement of single-stranded slat extensions on the “west” and “south” sides of the ribbon respectively. Through a combination of different extension lengths and staggers of the slats, the cooperative binding sites (e.g., comprising 5-6 nucleotides in length) on these extensions produce tochold domains in a direction perpendicular to the strands themselves. Single-stranded DNA (ssDNA) “cut-slats” or “slat strands” comprise domains that are complementary to these tochold domains, as well as the subsequent arrangement of x-slats and y-slats. Thus, slat strands can invade the ribbon via tochold-mediated strand displacement (TMSD), and are collectively able to entirely “cut” the ribbon in two. Thus, in some embodiments, ribbon scission (cleavage or cutting of the crisscross ribbon) of the crisscross ribbon occurs through the use of tochold-mediated strand displacement (TMSD). This results in two growth fronts, doubling the amount of possible ribbon assembly. See, for example, FIGS. 1A-1E.

Since both the crisscross ribbon(s) and slat strands are present in a one-pot reaction, in some embodiments, the growth/scission cycle constantly repeats, resulting in an exponential amplification process that generates many short ribbon fragments from a single seed. See, for example, right panel of FIG. 2A, which provides a TEM image of crisscross ribbon(s) in presence of slat strands (e.g., cut-slats and growth-slats). Given that the process of scission via TMSD is a random walk, the speed of the process can be biased towards more cutting by tuning the relative energetics of cut-slat binding as compared to growth-slat binding, for example by adjusting tochold length (e.g., length of tochold domain can comprise or consist of 3-20 nucleotides), guanine-cytosine (GC) content, the introduction of unpaired nucleotides, wobble base-pairs (e.g., guanine-thymine (GT) wobble base-pairs), or deletions.

In some embodiments, ribbon scission of the crisscross ribbon occurs via alternative methods that can be used alone or in combination with TMSD. In some embodiments, ribbon scission of the crisscross ribbon occurs via any combination of methods as described herein.

In some embodiments, ribbon scission is mediated by gamma cut slats. A gamma cut slat is a strand that represents the fusion between an x-slat and a y-slat. In some embodiments, ribbon scission mediated by gamma cut slats provides specific recruitment of the cut slat to a desired location (e.g., an interaction between an x-slat and a y-slat of the crisscross ribbon).

In some embodiments, ribbon scission is mediated by engineering one or more restriction sites (e.g., endonuclease restriction site for an endonuclease restriction enzyme) into a slat strand. Engineering one or more restriction sites into a slat strand can, in some embodiments, provide a mechanism to cleave double-stranded nucleic acids (e.g., double-stranded nucleic acids that form only when the slat strand binds to a single-stranded extension of a strand of the crisscross ribbon) but not cleave the single-stranded slat strand (e.g., when not bound to a single-stranded extensions of a strand of the crisscross ribbon). In some embodiments, a recognition site (e.g., for the restriction enzyme) is split across multiple slat strands.

In some embodiments, ribbon scission is mediated by inclusion of unnatural bases for accelerated scission (e.g., inclusion of unnatural bases in either or both of the slat strands and strands of the crisscross ribbon). For example, in some embodiments, phosphorothioate linkages can be included in the strands of the crisscross ribbon (e.g., to weaken the binding interactions between the first set of strands and the second set of strands). In some embodiments, modified bases can be included in slat strands to strengthen the binding interaction between a slat strand and a strand of the crisscross ribbon. A modified base may include a locked nucleic acid, diaminopurine, 2′-O-methyl nucleotide, 2′-fluoro nucleotide, 5-methyl-cytosine, and others known in the art.

In some embodiments, ribbon scission is mediated by small molecules that can function to accelerate scission without major deceleration of growth (“small molecule chaperones”). For example, a small molecule chaperone can be included or introduced into a reaction mixture comprising a set of strands that form a crisscross ribbon and slat strands in order to accelerate scission. Examples of small molecule chaperones include crowding agents, polyethylene glycols such as PEG-200, and molecules as described in Chao, S. et al., DNA Self-assembly Catalyzed by Artificial Agents, Scientific Reports volume 7, Article number: 6818 (2017); and Volodin, A. et al., Polycationic ligands of different chemical classes stimulate DNA strand displacement between short oligonucleotides in a protein-free system, Biopolymers. 2016 September; 105 (9): 633-41. doi: 10.1002/bip.22859.

In some embodiments, ribbon scission is mediated by the use of pH responsive elements that can be used to trigger scission (e.g., at low pH). In some embodiments, a pH responsive element can be included in the strands that form a crisscross ribbon and/or slat strands. In some embodiments, a pH responsive element can be included in a reaction mixture. In some embodiments, strands that form a crisscross ribbon can incorporate sequences that form i-motif self-structures at low pH (e.g., in order to weaken interactions between the first set of strands and the second set of strands). In some embodiments, a pH responsive element is as described in Dong, Y. et al., DNA nanotechnology based on i-motif structures., Acc Chem Res. 2014 Jun. 17; 47 (6): 1853-60. doi: 10.1021/ar500073a. Epub 2014 May 20. In some embodiments, ribbon scission is mediated by inclusion of a photoacid such as spiropyran. In some embodiments, ribbon scission is mediated by inclusion of a pH responsive element as described in Ryssy, J. et al., Light-Responsive Dynamic DNA-Origami-Based Plasmonic Assemblies., Angew Chem Int Ed Engl. 2021 Mar. 8; 60 (11): 5859-5863. doi: 10.1002/anic.202014963. Epub 2021 Feb. 16.

In some embodiments, ribbon scission is mediated by engineering crosslinking or ligation junctions (e.g., junctions that can be ligated by enzymatic ligases) into the strands that form a crisscross ribbon and/or slat strands. For example, ligation junctions can be engineered to form on the ends of a crisscross ribbon after the binding of a slat strand to the ribbon. In such embodiments, the isolated slat strands would be poor substrates for a ligase (e.g., T4 DNA ligase). In some embodiments, ligation could increase the length of the slat extension after slat strands have incorporated which can assist to speed up amplification. In some embodiments, methods to engineer crosslinking junctions are as described in Nakamura, S. et al., Photochemical Acceleration of DNA Strand Displacement by Using Ultrafast DNA Photo-crosslinking., Chembiochem. 2017 Oct. 18; 18 (20): 1984-1989. doi: 10.1002/cbic.201700430. Epub 2017 Aug. 29.

In some embodiments, ribbon scission is mediated by mechanical rupturing of crisscross ribbons (e.g., to break ribbons into smaller pieces) in order to increase the number of growth fronts available for binding to a slat strand. In some embodiments, mechanical rupturing can be performed using sonication. In some embodiments, mechanical rupturing can be performed using a method as described in Carlier, M. F. et al. Polymerization of ADP-actin and ATP-actin under sonication and characteristics of the ATP-actin equilibrium polymer., J Biol Chem. 1985 Jun. 10; 260 (11): 6565-71.

In some embodiments, ribbon scission is mediated by engineering photothermal elements into strands that form a crisscross ribbon and/or slat strands (e.g., to produce localized heating). For example, binding of gold nanoparticles to specific points along the crisscross ribbon can allow for photo excitation to generate local increase in heat, thereby denaturing the ribbons at those specific points (e.g., but not globally denaturing the ribbon). In some embodiments, engineering photothermal elements into strands that form a crisscross ribbon and/or slat strands can be performed using methods as described in Hastman, D. A., et al., Femtosecond Laser Pulse Excitation of DNA-Labeled Gold Nanoparticles: Establishing a Quantitative Local Nanothermometer for Biological Applications. ACS Nano. 2020 Jul. 28; 14 (7): 8570-8583. doi: 10.1021/acsnano.0c02899. Epub 2020 Jul. 17.

In some embodiments, ribbon scission is mediated by polymerase-based scission. For example, in some embodiments, scission of ribbons is mediated by strand-displacing polymerases such as Bst polymerase.

In some embodiments, ribbon scission is mediated by engineering thermoactivated slat strands. In some embodiments, slat strands are engineered to be sequestered in inactive complexes at lower temperatures, and then released to base pair with strands of a crisscross ribbon at higher temperatures. In some embodiments, thermoactivated slat strands can be engineered using methods as described in Hahn, J. and Shih, W. Thermal cycling of DNA devices via associative strand displacement., Nucleic Acids Res. 2019 Nov. 18; 47 (20): 10968-10975. doi: 10.1093/nar/gkz844.

In some embodiments, ribbon scission is mediated by engineering nicking sites to create tochold into strands that form a crisscross ribbon and/or slat strands. In some embodiments, nicking sites are included into slat strands such that the slat strand functions as a more potent tochold displacing strand following nicking of the slat strand than prior to nicking.

In some embodiments, ribbon scission is mediated by exonucleases. For example, in some embodiments, exonucleases can be used to initiate processive degradation of strands (e.g., slat strands) only after those strands have been incorporated into a crisscross ribbon.

In some embodiments, tochold domain formed by the single-stranded extensions of nucleic acids in a crisscross ribbon comprises 2-15, 2-20, 2-25, 2-30, 2-35, 2-40, 2-45 or 2-50 cooperative binding sites. In some embodiments, the number of cooperative binding sites is 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100. In some embodiments, a short tochold domain comprises 2 cooperative binding sites. In some embodiments, a medium tochold domain comprises 3 cooperative binding sites. In some embodiments, a long tochold domain comprises 4 cooperative binding sites.

A cooperative binding site of a tochold domain may have a length of 5-40, 5-30, 5-20, 5-10, 5-15, 10-50, 10-40, 10-30, 10-20, 30-50, 30-40, or 40-50 nucleotides. In some embodiments, a cooperative binding site has a length of 5, 10, 15, 20, 25, 30, 35, 40, 45, or 50 nucleotides. In some embodiments, a cooperative binding site may have a length of 4, 5, 6, 7, 8, 9 or 10 nucleotides.

In some embodiments, the total guanine-cytosine (GC) content of the nucleic acid layers of a crisscross ribbon comprises 20-80% total GC. In some embodiments, the total guanine-cytosine (GC) content of the nucleic acid layers of a crisscross ribbon comprises 20-40%, 30-40%, 35-50%, 40-60%, 50-70%, 60-80%, or 70-80% total GC. In some embodiments, the total guanine-cytosine (GC) content of the slat strands comprises 20-80% total GC. In some embodiments, the total guanine-cytosine (GC) content of the slat strands comprises 20-40%, 30-40%, 35-50%, 40-60%, 50-70%, 60-80%, or 70-80% total GC.

In some embodiments, a crisscross ribbon comprises unpaired nucleotides between the two layers. In some embodiments, a crisscross ribbon comprises 1-10 unpaired nucleotides along a stretch of nucleotides (e.g., a stretch of 10-20 nucleotides). In some embodiments, a crisscross ribbon comprises 1, 2, 3, 4, 5, 6, 7, 8, 9, or 10 unpaired nucleotides along a stretch of 10-20 nucleotides (e.g., 10, 11, 12, 13, 14, 15, 16, 17, 18, 19, or 20 nucleotides). In some embodiments, 1-20%, 1-10%, 1-5%, 2-10%, 2-5% or 3-6% of nucleotides of a first layer of a crisscross ribbon are unpaired. An unpaired nucleotide may result from a nucleotide deletion in one of the two layers.

A slat strand is capable, in some embodiments, of binding to multiple strands of a crisscross ribbon. In some embodiments, the slat strand(s) bind to a single-stranded extension (‘tochold domain’) at one or both of the terminal ends of each nucleic acid strand of the first layer of strands that make up a crisscross ribbon. In some embodiments, a slat strand is complementary (e.g., 100% complementary) to a single-stranded extension. In some embodiments, a first nucleic acid strand (e.g., a slat strand) is “complementary” to a second nucleic acid strand (e.g., a single-stranded extension) if it base-pairs or binds to the second nucleic acid strand form a double-stranded nucleic acid molecule via Watson-Crick interactions and/or non-Watson-Crick base pairing (also referred to as hybridization). In some embodiments, two nucleic acid strands are complementary to one another if at least 70%, 75%, 80%, 85%, 90%, 95%, 99%, or 100% of the nucleobases across the length of one of the first nucleic acid strand are base-paired to nucleobases of the second nucleic acid strand.

In some embodiments, a binding interaction between a tochold domain and a slat strand comprises unpaired nucleotides. In some embodiments, a binding interaction between a tochold domain and a slat strand comprises 1-10 unpaired nucleotides along a stretch of nucleotides (e.g., a stretch of 10-20 nucleotides). In some embodiments, a binding interaction between a tochold domain and a slat strand comprises 1, 2, 3, 4, 5, 6, 7, 8, 9, or 10 unpaired nucleotides along a stretch of 10-20 nucleotides (e.g., 10, 11, 12, 13, 14, 15, 16, 17, 18, 19, or 20 nucleotides). In some embodiments, 1-20%, 1-10%, 1-5%, 2-10%, 2-5% or 3-6% of nucleotides of tochold domain are unpaired when the tochold domain is binding to a slat strand. An unpaired nucleotide may result from a nucleotide deletion in the tochold or the slat strand.

In some embodiments, a tochold domain comprises or consists of 3-20 nucleotides in length. In some In some embodiments, a tochold domain comprises or consists of 3-15, 3-12, 3-10, 4-10, 5-10, 5-12, 6-20, or 7-20 nucleotides in length. embodiments, a tochold domain comprises or consists of 3, 4, 5, 6, 7, 8, 9, 10, 11, 12, 13, 14, 15, 16, 17, 18, 19, or 20 nucleotides in length.

In some embodiments, a crisscross ribbon comprises wobble base-pairs between the two layers of the crisscross ribbon. In some embodiments, a crisscross ribbon comprises 1-10 wobble base-pairs along a stretch of nucleotides (e.g., a stretch of 10-20 nucleotides). In some embodiments, a crisscross ribbon comprises 1, 2, 3, 4, 5, 6, 7, 8, 9, or 10 wobble base-pairs along a stretch of 10-20 nucleotides (e.g., 10, 11, 12, 13, 14, 15, 16, 17, 18, 19, or 20 nucleotides). In some embodiments, 1-20%, 1-10%, 1-5%, 2-10%, 2-5% or 3-6% of nucleotides of a first layer of a crisscross ribbon comprise wobble base-pairs.

In some embodiments, a binding interaction between a tochold domain and a slat strand comprises wobble base-pairs. In some embodiments, a binding interaction between a tochold domain and a slat strand comprises 1-10 wobble base-pairs along a stretch of nucleotides (e.g., a stretch of 10-20 nucleotides). In some embodiments, a binding interaction between a tochold domain and a slat strand comprises 1, 2, 3, 4, 5, 6, 7, 8, 9, or 10 wobble base-pairs along a stretch of 10-20 nucleotides (e.g., 10, 11, 12, 13, 14, 15, 16, 17, 18, 19, or 20 nucleotides). In some embodiments, 1-20%, 1-10%, 1-5%, 2-10%, 2-5% or 3-6% of nucleotides of tochold domain form a wobble base-pair when the tochold domain is binding to a slat strand.

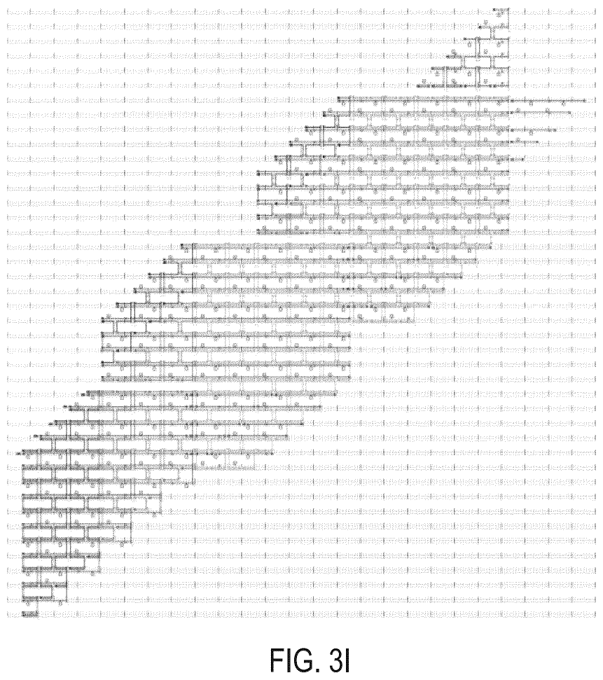

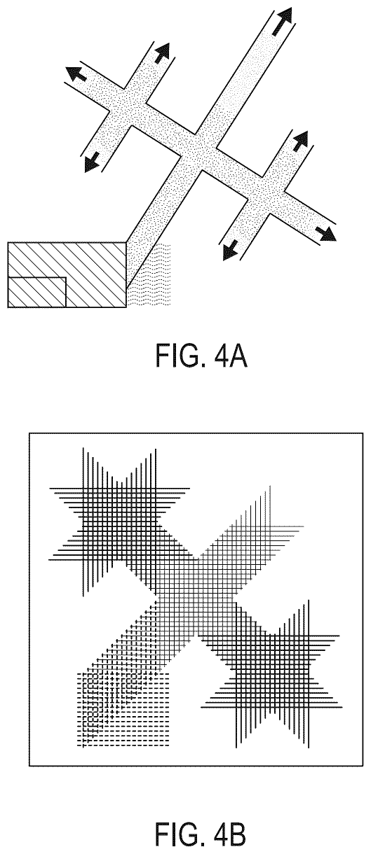

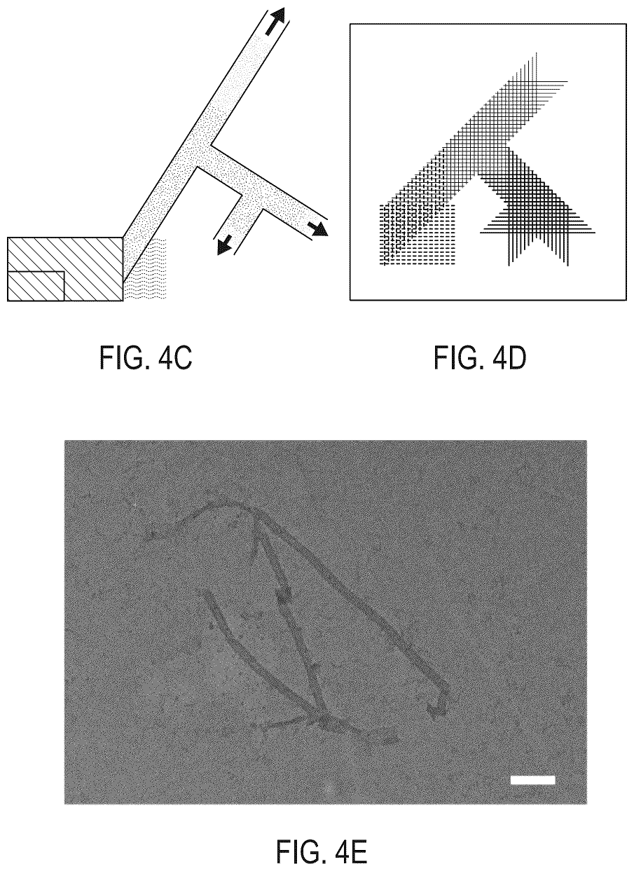

In some embodiments, crisscross cooperative assembly further comprises dendrimeric ribbon assembly. For example, instead of binding to cut-slats or slat strands, single-stranded extensions on layers of nucleic acids of a crisscross ribbon (e.g., growth-slats) can also be programmed to bind additional generations of layers of nucleic acids or growth-slats to produce branched structures. In some embodiments, this produces a one-to-one conversion of seed to dendrimeric ribbon. Sec, e.g., FIGS. 4A-4E. While the degree of amplification may be closer to cubic than exponential (due to geometric constraints), such an approach could allow for digital counting of structures via fluorescence microscopy.

### Nucleic Acid Nanostructures

A “nucleic acid nanostructure,” including a “DNA nanostructure” or a “crisscross ribbon,” refers to a nanostructure (e.g., a structure that is between 0.1 nm and 1 μm (e.g., 0.1 nm and 100 nm) in each spatial dimension, e.g., 1D, 2D or 3D) that is rationally designed to self-assemble (is programmed) into a pre-determined, defined shape that would not otherwise assemble in nature. The use of nucleic acids to build nanostructures is enabled by strict nucleotide base pairing rules (e.g., A binds to T, G binds to C, A does not bind to G or C, T does not bind to G or C), which result in portions of strands with complementary base sequences binding together to form strong, rigid structures. This allows for the rational design of nucleotide base sequences that will selectively assemble (self-assemble) to form nanostructures.

While the term “nanostructure” is used throughout the present disclosure with reference to end structures (e.g., DNA nanostructures), it should be understood that unless stated otherwise the methods described herein may be used to assemble any one of a nanostructure, macrostructure, or microstructure. Thus, in any one of the embodiments described herein, the term “nanostructure” encompasses “macrostructure” and “microstructure” unless stated otherwise. It should also be understood, however, that “macrostructure” and/or “microstructure” may be specifically excluded from any one of the embodiments described herein.

Examples of nucleic acid (e.g., DNA) nanostructures include, but are not limited to, DNA origami structures, in which a long scaffold strand (e.g., at least 500 nucleotides in length) is folded by hundreds (e.g., 100, 200, 200, 400, 500 or more) of short (e.g., less than 200, less than 100 nucleotides in length) auxiliary strands into a complex shape (Rothemund, P. W. K. Nature 440, 297-302 (2006); Douglas, S. M. et al. Nature 459, 414-418 (2009); Andersen, E. S. et al. Nature 459, 73-76 (2009); Dictz, H. et al. Science 325, 725-730 (2009); Han, D. et al. Science 332, 342-346 (2011); Liu, Wet al. Angew. Chem. Int. Ed. 50, 264-267 (2011); Zhao, Z. et al. Nano Lett. 11, 2997-3002 (2011); Woo, S. & Rothemund, P. Nat. Chem. 3, 620-627 (2011); Tørring, T. et al. Chem. Soc. Rev. 40, 5636-5646 (2011)). Other more modular strategies have also been used to assemble DNA tiles (Fu, T. J. & Sccman, N. C. Biochemistry 32, 3211-3220 (1993); Winfrec, E. et al. Nature 394, 539-544 (1998); Yan, H. et al. Science 301, 1882-1884 (2003); Rothemund, P. W. K. et al. PLOS Biol. 2, c424 (2004); Park, S. H. et al. Angew. Chem. Int. Ed. 45, 735-739 (2006); Schulman, R. & Winfrec, E. Proc. Natl Acad. Sci. USA 104, 15236-15241 (2007); Hc, Y. et al. Nature 452, 198-201 (2008); Yin, P. et al. Science 321, 824-826 (2008); Sharma, J. et al. Science 323, 112-116 (2009); Zheng, J. P. et al. Nature 461, 74-77 (2009); Lin, C. et al. ChemPhysChem 7, 1641-1647 (2006)) or RNA tiles (Chworos, A. et al. Science 306, 2068-2072 (2004); Delebecque, C. J. et al. Science 333, 470-474 (2011)) into periodic (Winfree, E. et al., Nature 394, 539-544 (1998); Yan, H. et al. Science 301, 1882-1884 (2003); Chworos, A. et al. Science 306, 2068-2072 (2004); Delebecque, C. J. et al. Science 333, 470-474 (2011)) and algorithmic (Rothemund, P. W. K. et al. PLOS Biol. 2, c424 (2004)) two-dimensional lattices (Seeman, N. C. J. Theor. Biol. 99, 237-247 (1982); Park, S. H. et al. Angew. Chem. Int. Ed. 45, 735-739 (2006)), extended ribbons (Schulman, R. & Winfrec, E. Proc. Natl Acad. Sci. USA 104, 15236-15241 (2007); Yin, P. et al. Science 321, 824-826 (2008)) and tubes (Yan, H. et al. Science 301, 1882-1884 (2003); Yin, P. et al. Science 321, 824-826 (2008); Sharma, J. et al. Science 323, 112-116 (2009)), three-dimensional crystals (Zheng, J. P. et al. Nature 461, 74-77 (2009)), polyhedral (Hc, Y. et al. Nature 452, 198-201 (2008)) and simple finite two-dimensional shapes (Chworos, A. et al. Science 306, 2068-2072 (2004); Park, S. H. et al. Angew. Chem. Int. Ed. 45, 735-739 (2006)).

Thus, crisscross cooperative assembly building blocks (e.g., nucleating nanostructures and subsets of nanostructures) may be one of a number of nucleic acid nanostructure shapes, including, but not limited to, rods/tubes, sheets, ribbons, lattices, cubes, spheres, polyhedral, or another two-dimensional or three-dimensional shape. In some embodiments, a nanostructure has junction(s), branch(es), crossovers, and/or double-crossovers formed by nucleotide base pairing of two or more nucleic acid strands (see, e.g., Mao, C. PLOS Biology, 2 (12), 2036-2038, 2004).

In some embodiments, a nucleic acid nanostructure is a seed molecule.

The versatile and stable nature of DNA origami enables the construction of various individual architectures that can be designed in a particular way, to facilitate to cooperative assembly of larger structures. In one example, each component is a separately folded DNA-origami structure.

A nucleic acid (e.g., DNA) slat is a slat-shaped nanostructure that is composed of DNA. A slat may be an antiparallel-crossover single-stranded slat (AXSSS) comprising single strands that cross a partnering single strand only once. Also provided herein are paranemic crossover slats that include a pair of strands that cross another pair of strands.

Similar to the larger scale DNA-origami crisscross cooperative assembly, single-stranded DNA can be used to achieve cooperative assembly of higher order structures. In order to achieve this, drones and workers are replaced with oligonucleotides of various lengths (depending on the proposed architecture) that can assemble onto a DNA-origami queen nucleation site or onto a single stranded DNA catenane structure. In some embodiments, ring structures are comprised of single-stranded DNA that has exposed binding sites for nucleic acids. In another example, the components are folded into a DNA origami barrel queen. The scaffold can be tiled with extended DNA slats (slats) capable of seeding further DNA slats, leading to growth of the structure. In some embodiments, the DNA slats work in two steps: first, folding the origami queen site (for example, mixing M13 scaffold and staple strands), and second, mixing the crude DNA origami queen reaction with DNA slats, leading to growth of the structure. Varying salt concentrations, temperatures, and DNA slat concentration can alter the binding energy of the various sub-components, leading to reversible or irreversible binding, for example.

Typically, nucleic acid nanostructures do not contain coding sequences (sequences that code for a full length mRNA or protein), thus, nucleic acid nanostructures do not contain a promoter or other genetic elements that control gene/protein expression. An individual single-stranded nucleic acid (e.g., DNA strand or RNA strand without secondary structure), or an individual double-stranded nucleic acid (e.g., without secondary structure), for example, double helices found in nature or produced synthetically or recombinantly (e.g., such as a plasmid or other expression vector), are specifically excluded from the definition of a nucleic acid nanostructure.

Nanostructures, in some embodiments, have a void volume, which is the combine volume of space between nucleic acids that form a nanostructures. It should be understood that “space” includes fluid-filled space. Thus, a nanostructure in solution, have a void volume of 25% may include 75% nucleic acids and 25% reaction buffer (filling the 25% void volume of the nanostructure). In some embodiments, a nanostructure in solution, e.g., in reaction buffer, may have a void volume of at least 10% (e.g., 10-90%, 10-80%, 10-70%, 10-60%, 10-50%, 10-40%, or 10-30%), at least 20% (e.g., 20-90%, 20-80%, 20-70%, 20-60%, 20-50%, 20-40%, or 20-30%), at least 30%, (e.g., 30-90%, 30-80%, 30-70%, 30-60%, 30-50%, or 30-40%), at least 40% (e.g., 40-90%, 40-80%, 40-70%, 40-60%, or 40-50%), at least 50% (e.g., 50-90%, 50-80%, 50-70%, or 50-60%), at least 60% (e.g., 60-90%, 60-80%, or 60-70%), at least 70% (e.g., 70-90% or 70-80%), or at least 80% (e.g., 80-90%). In some embodiments, a nanostructure has a void volume of 10%, 15%, 20%, 25%, 30%, 35%, 40%, 45%, 50%, 55%, 60%, 65%, 70%, 75%, 80%, 85%, or 90%.

A “nucleic acid nanorod,” including a “DNA nanorod” is a nucleic acid (e.g., DNA) nanostructure in the shape of a rod. A nanorod is a three-dimensional cylindrical shape having a length longer than its diameter. In some embodiments, a nucleic acid nanorod comprises six helix bundles. For example, six DNA double helices may be connected to each other at two crossover sites. DNA double helices with 10.5 nucleotide pairs per turn facilitate the programming of DNA double crossover molecules to form hexagonally symmetric arrangements when the crossover points are separated by seven or fourteen nucleotide pairs (see, e.g., Mathieu F. et al. Nano Lett. 5 (4), 661-664 (2005)). Other methods of assembling nucleic acid nanorods (also referred to as nanotubes) may be used (see, e.g., Feldkamp, U. et al. Angew. Chem. Int. Ed. 45 (12), 1856-1876 (2006); Hariri A. et al. Nature Chemistry, 7, 295-300 (2015)).

The length and diameter of a nanorod (or other nanostructure) may vary. In some embodiments, a nanorod (or other nanostructure) has a length of 10-100 nm, or 10-500 nm. For example, a nanorod may have a length of 10-500 nm, 10-400 nm, 10-300 nm, 10-200 nm, 10-100 nm, 10-90 nm, 10-80 nm, 10-70 nm, 10-60 nm, 10-50 nm, 10-30 nm, or 10-20 nm. In some embodiments, a nanorod has a length of 100-500 nm, 200-500 nm, or 300-500 nm. In some embodiments, a nanorod has a length of 100 nm, 150 nm, 200 nm, 250 nm, 300 nm, 350 nm, 400 nm, 450 nm or 500 nm. In some embodiments, a nanorod has a length of 10, 15, 20, 25, 30, 35, 40, 45, 50, 55, 60, 65, 70, 75, 80, 85, 90, 95 or 100 nm. In some embodiments, the length of a nanorod (or other nanostructure) is longer than 100 nm (e.g., 100-1000 nm), or shorter than 10 nm (e.g., 1-10 nm). In some embodiments, a nanorod (or other nanostructure) has a diameter of 5-90 nm. For example, a nanorod may have a diameter of 5-80 nm, 5-70 nm, 5-60 nm, 5-50 nm, 5-30 nm, 5-20 or 5-10 nm. In some embodiments, a nanorod has a diameter of 5, 10, 15, 20, 25, 30, 35, 40, 45, 50, 55, 60, 65, 70, 75, 80, 85 or 90 nm. In some embodiments, the diameter of a nanorod is longer than 9 nm, or shorter than 5 nm. Thus, in some embodiments, a nanorod (or other nanostructure) has a circumference of 15-300 nm (C≈3.14×d).

A nucleic acid nanostructure, such as a nanorod, is considered “elongated,” if the length of the nanostructure is longer than its width/diameter (e.g., by at least 10%, 20%, 25%, 50%, 100%, or 200%).

Nucleic acid nanostructures are typically nanometer-scale structures (e.g., having lengths of 1 to 1000 nanometers). In some embodiments, however, the term “nanostructure” herein may include micrometer-scale structures (e.g., assembled from more than one nanometer-scale or micrometer-scale structure). In some embodiments, a nanostructure has a dimension (e.g., length or width/diameter) of greater than 500 nm or greater than 1000 nm. In some embodiments, a nanostructure has a dimension of 1 micrometer to 2 micrometers. In some embodiments, a nanostructure has a dimension of 10 to 500 nm, 10 to 450 nm, 10 to 400 nm, 10 to 350 nm, 10 to 300 nm, 10 to 250 nm, 10 to 200 nm, 10 to 150 nm, 10 to 100 nm, 10 to 50 nm, or 10 to 25 nm. In some embodiments, the nanostructure has a dimension of 500 to 450 nm, 500 to 400 nm, 500 to 350 nm, 500 to 300 nm, 500 to 250 nm, 500 to 200 nm, 500 to 150 nm, 500 to 100 nm, 500 to 50 nm, or 500 to 25 nm. In some embodiments, the nanostructure has a dimension of 10, 25, 50, 75, 100, 150, 200, 250, 300, 350, 400, 450, or 500 nm.

A nucleic acid nanostructure is considered to “self-assemble.” Bottom up, self-assembly refers to the process by which molecules adopt a defined arrangement without guidance or management from an outside source. Although, it should be understood that with synthetic nucleic acid self-assembly, as provided herein, the nucleotide base sequences that guide assembly of nucleic acids are artificially designed, and the corresponding nucleic acids are accordingly synthesized by an outside source, such as one of skill in the art (using, for example, standard nucleic acid synthesis techniques). That is, one of ordinary skill in the art can ‘program’ nucleotide base sequences within a single nucleic acid strand or between two difference nucleic acid strands to selectively bind to each other in solution based on a strict set of nucleotide base pairing rules (e.g., A binds to T, G binds to C, A does not bind to G or C, T does not bind to G or C). Self-assembly may be intramolecular (folding) or intermolecular.

The nanostructures assembled from smaller nucleic acid-based are “rationally designed.” A nanostructure, as discussed above, does not assemble in nature. Nucleic acid strands for use in crisscross cooperative assembly are ‘programmed’ such that among a specific population of strands, complementary nucleotide base sequences within the same strand or between two different strands bind selectively to each other to form a complex, user-defined structure, such as a rod/tube, ribbon, lattice, sheet, polyhedral, cube, sphere, or other two-dimensional or three-dimensional shape. A nanostructure may have a regular shape (sides that are all equal and interior angles that are all equal) or an irregular shape (sides and angles of any length and degree).

### Methods of Crisscross Cooperative Assembly

Self-assembly of a nucleating nanostructure (e.g., a crisscross ribbon) and subsets of nanostructures occurs, in some embodiments, in a ‘one-pot’ reaction, whereby all elements of a crisscross cooperative assembly system as described herein are combined in a reaction buffer, and then the reaction buffer is incubated under conditions that result in self-assembly of all of the nucleic acid nanostructures.

Conditions that result in self-assembly of nucleic acids of a crisscross cooperative assembly reaction may vary depending on the size, shape, composition and number of nucleic acid strands in a particular reaction. Such conditions may be determined by one of ordinary skill in the art, for example, one who rationally designs/programs the nanostructures to self-assemble.

A crisscross cooperative assembly method may be performed at a variety of temperatures. In some embodiments, a crisscross cooperative assembly method is performed at room temperature (˜25° C.) or 37° C. A crisscross cooperative assembly method may be performed at a temperature lower than 25° C. or higher than 37° C. In some embodiments, a crisscross cooperative assembly method may be performed at a temperature between 20-60° C., 25-55° C., 25-50° C., 30-60° C., 30-50° C., 35-60° C., 35-45° C., 40-60° C., or 50-60° C.

The salt concentration of the reaction buffer in which a crisscross cooperative assembly reaction is performed may also vary. In some embodiments, the reaction buffer comprises MgCl₂ salt at a concentration of 1 mM-10 mM (e.g., 1 mM, 2 mM, 3 mM, 4 mM, 5 mM, 6 mM, 7 mM, 8 mM, 9 mM or 10 mM), 5-50 mM, 5-25 mM, or 10-20 mM. In some embodiments, the reaction buffer comprises NaCl at a concentration of 100 mM-500 mM (e.g., 100 mM, 200 mM, 300 mM, 400 mM or 500 mM). In some embodiments, a crisscross cooperative assembly method is performed under high-salt conditions. Thus, in some embodiments, the reaction buffer comprises MgCl₂ salt at a concentration of at least 20 mM (e.g., 20-500 mM, or 20-200 mM). In some embodiments, the reaction buffer comprises NaCl at a concentration of at least 1 M (e.g., 1-2 M, 1-3 M, 1-4 M, or 1-5 M).

In any given reaction, the number of initial nanostructures or set of nucleic acid strands exceeds the number of nucleating nanostructures or set of nucleic acid strands. Thus, in some embodiments, the ratio of nucleating nanostructure to non-nucleating nanostructure (e.g., a drone from an initial subset, or a worker from a subsequent subset) is 1:10-1:10¹² (trillion). For example, the ratio of nucleating nanostructure to non-nucleating nanostructure may be 1:10-1:1000, 1:10-1:500, 1:10-1:100, 1:10-1:75, 1:10-1:50, or 1:10-1:25. In some embodiments, the ratio of nucleating nanostructure to non-nucleating nanostructure is 1:1000, 1:500, 1:100, 1:90, 1:80, 1:70, 1:60, 1:50, 1:40, 1:30, 1:20 or 1:10. In some embodiments, the ratio of a first (or initial) set of nucleic acid strands to a second set of nucleic acid strands is 1:10 1:10¹² (trillion). For example, the ratio of a first (or initial) set of nucleic acid strands to a second set of nucleic acid strands may be 1:10-1:1000, 1:10-1:500, 1:10-1:100, 1:10-1:75, 1:10-1:50, or 1:10-1:25. In some embodiments, the a first (or initial) set of nucleic acid strands to a second set of nucleic acid strands is 1:1000, 1:500, 1:100, 1:90, 1:80, 1:70, 1:60, 1:50, 1:40, 1:30, 1:20 or 1:10.

In some embodiments, a crisscross cooperative assembly reaction is incubated for 2-96 hours. For example, a crisscross cooperative assembly reaction may be incubated for 2-24 hours, 2-30 hours, 2-36 hours, 2-42 hours, 2-48 hours, 2-54 hours, 2-60 hours, 2-66 hours, 2-72 hours, 2-78 hours, 2-84 hours, 2-90 hours, or 2-96 hours. In some embodiments, a crisscross cooperative assembly reaction is incubated for 3, 6, 9, 12, 15, 18, 21, 24, 27, 30, 33, 36, 39, 42, 45, 48, 51, 54, 57, 60, 63, 66, 69, or 72 hours. In some embodiments, a crisscross cooperative assembly reaction is incubated for 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70 or 72 hours.

A method described herein may, in some embodiments, be performed in the presence of an organic solvent. An organic solvent includes, but is not limited to, ethanol, methanol, hexanol, butanol, dichloromethane, and/or hexane.

The crisscross assembly methods described herein may be used to detect an environmental change in a sample. For example, in some embodiments, a method for detecting an environmental change comprises combining a set of nucleic acid strands that bind to each other to form a crisscross ribbon comprising a first layer of strands and a second layer of strands, wherein the strands of the first layer are nonparallel to and bound to the strands of the second layer, and slat strands, each of which binds to multiple strands of the first layer of strands that are bound to the strands of the second layer following an environmental change, thereby displacing the strands of the first layer from the strands of the second layer. An environmental change may be a pH change (e.g., a pH change such that a sample becomes acidic, more acidic, basic, or more basic compared to baseline), a temperature change (e.g., an increase in temperature of 1-40° C. or a decrease in temperature of 1-40° C.) or a change in the concentration of one or more metal ions (e.g., a change in concentration by at least 10 μM, 100 μM, 500 μM, 1 mM, 5 mM, 10 mM, or 100 mM of a metal ion, e.g., a divalent cation such as Ca²⁺ Mg²⁺).

### Detection of a Biomarker

In some embodiments, the crisscross assembly products may be used for detecting a selected biomarker (e.g., a biomolecule comprising seed strands to which strands of a first layer and/or second layer of a crisscross slat are capable of binding) using a variety of different mechanisms and the systems described herein. For example, in such systems, the presence of a biomolecule can be used to trigger crisscross assembly, which can then be detected (visualized), indicating the presence of the biomolecule.

The biomarker may be detected by incubating nucleic acids and slat strands in the presence of the biomarker such that a nucleic acid nanostructure (e.g., a branched nucleic acid nanostructure) is produced. In some embodiments, the biomarker is detected by combining in a reaction mixture (a) a set of nucleic acid strands that bind to each other to form a crisscross ribbon comprising a first layer of strands and a second layer of strands, wherein the strands of the first layer are nonparallel to and bound to the strands of the second layer, (b) a biomolecule comprising seed strands to which strands of the first layer and/or second layer of the crisscross slat are bound, wherein the biomolecule seed is representative of the biomarker; and (c) slat strands, each of which binds to multiple strands of the crisscross ribbon that are bound to the seed strands; and incubating the reaction mixture under conditions that result in production of a branched nucleic acid nanostructure, wherein visualization of the nanostructure enables detection of the biomarker.

The biomarker may be detected using a ring system. In some embodiments, a large DNA ring (“host ring”), single-stranded DNA, may be split to incorporate a biomarker capture site (analyte test site) to bind macromolecules in biological samples. The DNA ring loops through and encloses a number of discrete, separate “guest” rings, which are single-stranded DNA and function as catenane queens, so that the guest rings are catenated on the host ring, similar to individual beads on a bracelet. In some embodiments, the guest rings are independently formed from separate single-stranded nucleic acids, while in other embodiments, the guest rings are formed from a long single nucleic acid strand assembled into multiple (e.g., vertically stacked) rings. The number of guest rings can be 2, 3, 4, or 5 or more. In embodiments, each guest ring (catenane queen) comprises binding sites for drone and worker oligonucleotides and is therefore capable of crisscross assembly. In embodiments, the plurality of catenated guest rings when in close proximity forms a catenane queen comprising binding sites (e.g., plug strands) for drone and worker nucleic acids and/or structures and is thus capable of crisscross assembly. The number of binding sites per guest ring can vary, and may be 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100. A biomarker test site, located near the biomarker capture site may also formed.

The presence of the biomarker, in some embodiments may be detected in mixtures, such as biological samples, as follows. First, a biological sample is mixed with a high concentration of the catenane queen, allowing macromolecules of interest bind the biomarker capture site. Then, a chemical reaction is used to reversibly cleave the biomarker capture site. Catenane queens not bound to the target biomarker will fall apart more quickly compared to those held together by the target biomarker. The remaining catenane queens in the test mixture are re-ligated at the biomarker test site. Subsequently, drones and workers are added to the test mixture to amplify remaining intact queens using readily observable micrometer-scale DNA structures. This system is modular, and the biomarker capture site may be customized to bind disease markers, including proteins or nucleic acid sequences.

Ultraspecific biosensors can also be created by adding a biomarker detection system to the multiple guest-ring (e.g., guest-loop) catenane systems with DNA slats. In this example, a barrel queen is used; however, other 3-dimensional shapes are also possible (e.g., sheets, blocks and dendrimers). An example of the production of a barrel queen (a rolled sheet) is described above.

Using a scaffold for DNA origami, for example an M13 scaffold and staple strands, a multiple guest-ring catenane system can be formed. For example, an eight-loop system is formed in a one-pot reaction. The number of loops (rings) can be varied, depending on the design of the system, and may be 2, 3, 4, 5, 6, 7, 8, 9, 10, 11, 12, 13, 14, 15, 16, 17, 18, 19, or 20 loops. Additional loops may be used. Unlike the system described above, the handles (elongated structures) and loops (rings) are all part of the same single-stranded DNA (e.g., M13 DNA); the handle structures are programmed to link together by specific staple strands (slats). The system is designed around the specific staple strands/slats; in the presence of biomarker, they hold the structure together and growth can occur from the parallel loops when drones and workers are added. In the absence of biomarker, the staple strands/slats release the structure, and no growth can occur as the queen falls apart and the binding sites are not close enough for nucleation and growth even in the presence of drones and workers. The presence of the structures can be detected using any one of the methods described above, or with any method known in the art.

DNA slats or other nucleic acids of a biosensor may be modified with one or more switchable bridges. A “switchable bridge” is a link between functional groups that forms or breaks in the presence of a particular agent (e.g., reaction agent or dissociation agent). Examples of switchable bridges include bonds formed via a “click chemistry” reaction (e.g., a between an azide and an alkyne), protein-protein binding (e.g., one or more antibodies binding to a target protein/antigen), a disulfide bond (between two thiols).

Thus, some aspects of the present disclosure provide a biosensor comprising (i) a first DNA slat comprising a first functional group (e.g., an azide or alkyne), a first binding partner (e.g., an antibody, aptamer or nanobody), and a second functional group (e.g., a thiol or nucleic acid), and (ii) a second DNA slat comprising a third functional group (e.g., a thiol or nucleic acid), a second binding partner (e.g., an antibody, aptamer or nanobody), and a fourth functional group (e.g., an azide or alkyne), wherein the first and fourth functional groups react in the presence of a reaction agent to form a link (e.g., a covalent link), wherein the first and fourth binding partners bind specifically to a biomarker of interest to form a link (e.g., non-covalent link), and wherein the second and third functional groups form a link (e.g., a covalent link) that breaks in the presence of a dissociation agent.

In some embodiments, a biosensor comprises a first DNA slat comprising an azide, an antibody, and a thiol group, and a second DNA slat comprising an alkyne, an antibody, and a thiol group, wherein antibody of (i) and the antibody of (ii) bind specifically to a biomarker of interest.

A “first biomarker binding partner” and a “second biomarker binding partner” are any molecules that bind to the same target biomarker to form a switchable bridge linking DNA slats to each other (via a non-covalent link). In some embodiments, the first and second biomarker binding partners are proteins or peptides. For example, the first and second biomarker binding partners may be antibodies that bind to different epitopes of the same antigen. Thus, in some embodiments, the first and second biomarker binding partners are antibodies (e.g., monoclonal, polyclonal, human, humanized or chimeric). In some embodiments, the first and second biomarker binding partners are antibody fragments (e.g., Fab, F(ab′) 2, Fc, scFv, or vhh). The biomarker binding partners may also be nanobodies or aptamers. Other protein-protein binding partners may be used.

A “first functional group” and a “fourth functional group” are functional groups that react with each other to form a link (bond, such as a covalent bond or a non-covalent bond), which forms a switchable bridge linking the DNA slats to each other. In some embodiments, this bridge is formed through a click chemistry (azide-alkyne cycloaddition) reaction (e.g., V. V. Rostovtsev, et al., Angew. Chem. Int. Ed., 2002, 41, 2596-2599; and F. Himo, et al. J. Am. Chem. Soc., 2005, 127, 210-216, each of which is incorporated herein by reference). Thus, in some embodiments, one of the first or fourth functional group is an azide, while the other of the first or fourth functional groups is an alkyne. For example, the first functional group may be azide, and the fourth functional group may be trans-cyclooctene (TCO). Other click chemistry functional groups may be used.

A “second functional group” and a “third functional group” are functional groups that react with each other to form a link (bond, such as a covalent bond or a non-covalent bond), which forms yet another switchable bridge linking the DNA slats to each other. This bridge breaks (dissociates) in the presence of a dissociation agent. A “dissociation agent” is an agent (e.g., chemical) that breaks the bond (e.g., covalent bond) between the second and third functional groups. In some embodiments, the second and third functional groups are thiol groups that react with each other to form a disulfide bridge. Thus, in some embodiments, the dissociation agent is dithiothreitol (DTT). In some embodiments, the concentration of DTT is 50 mM-200 mM. For example, the concentration of DTT may be 100 mM. Other functional groups may be used.

### EXAMPLES

### Example 1. Amplification Via Ribbon Scission

Nucleic acid strands were purchased from Integrated DNA technologies (IDT), and were purified by denaturing polyacrylamide gel electrophoresis. Crisscross ribbons were assembled overnight at 52° C., using concentrations of 0.5 μM/growth-slat, 1 μM/cut-slat, 14 mM MgCl₂, and 1 nM DNA origami seed. The growth-slats comprised a set of nucleic acid strands that bind to each other to form a crisscross ribbon comprising a first layer of strands and a second layer of strands, wherein the strands of the first layer comprise a single-stranded extension (which becomes a tochold domain) and were nonparallel to and complementary to the strands of the second layer. The cut-slats comprised slat strands, each of which was complementary to the growth-slats. The DNA origami seed comprised seed strands that were complementary to the growth-slats.

Following assembly, the crisscross ribbons were visualized using transmission electron microscopy (TEM). TEM grids were negatively stained using 2% aqueous uranyl formate. As shown in FIG. 2A (right panel), crisscross ribbon assembly with cut-slats, growth-slats, and the DNA origami seed resulted in formation of a large number of short fragments, relative to growth-slats only (left panel) (i.e., no assembly control). Scale bars are 500 nm.

### Example 2. Amplification Via Ribbon Scission with and without a Biomolecule Seed

Nucleic acid strands were purchased from Integrated DNA technologies (IDT) and were purified by denaturing polyacrylamide gel electrophoresis. Crisscross ribbons were assembled overnight at 54° C., using concentrations of 0.5 M/growth-slat, 0.5 μM/cut-slat, and 16 mM MgCl₂. In a first experiment, 1 nM DNA origami seed was also added (i.e., a biomolecule seed; ‘+S’). In a second experiment, no seed was added (‘−S′’). The growth-slats comprised a set of nucleic acid strands that bind to each other to form a crisscross ribbon comprising a first layer of strands and a second layer of strands, wherein the strands of the first layer comprise a single-stranded extension (which becomes a tochold domain) and were nonparallel to and complementary to the strands of the second layer. The cut-slats comprised slat strands, each of which was complementary to the growth-slats. The DNA origami seed comprised seed strands that were complementary to the growth-slats.

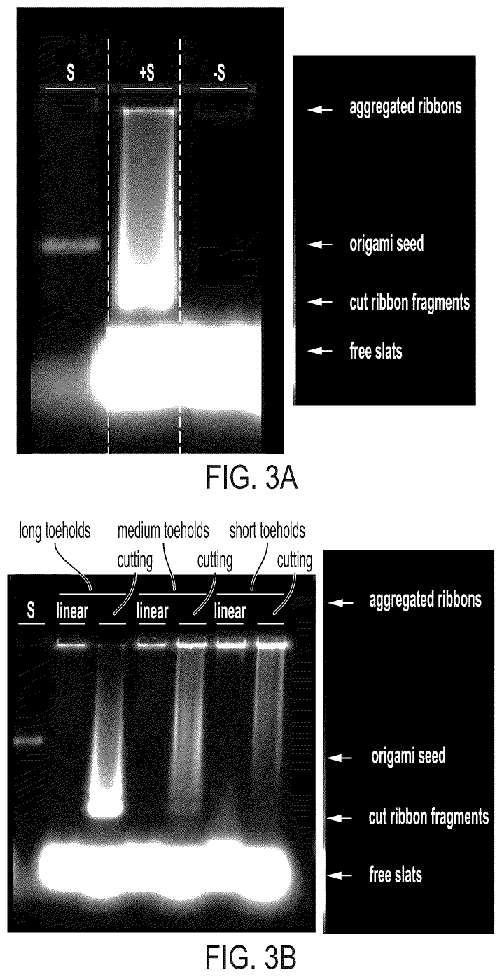

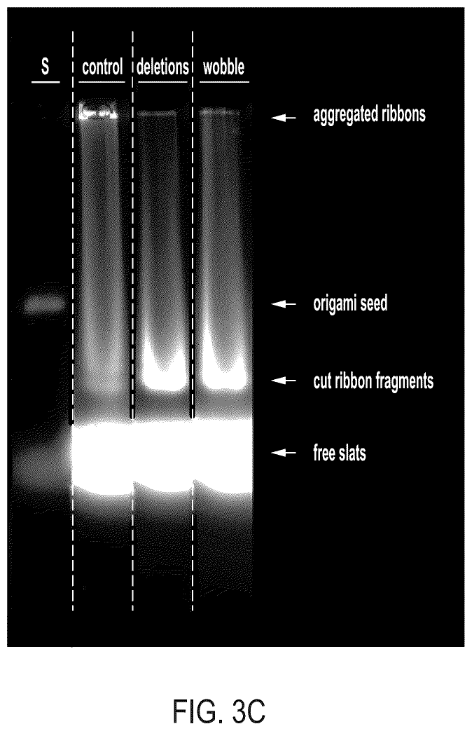

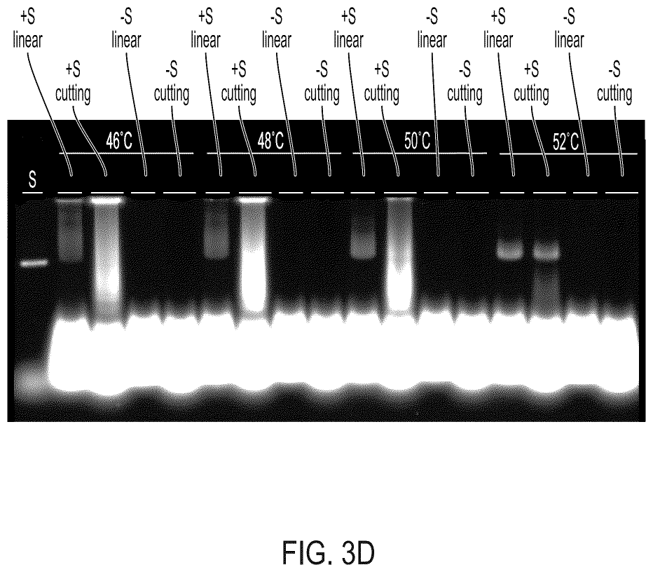

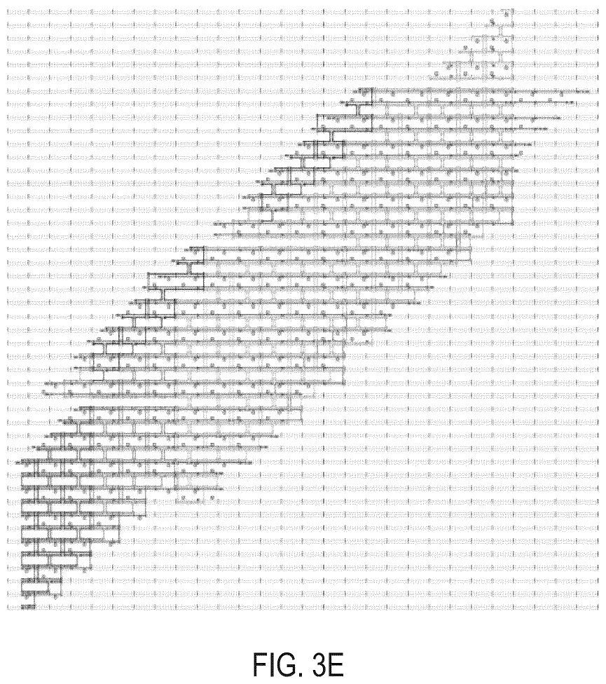

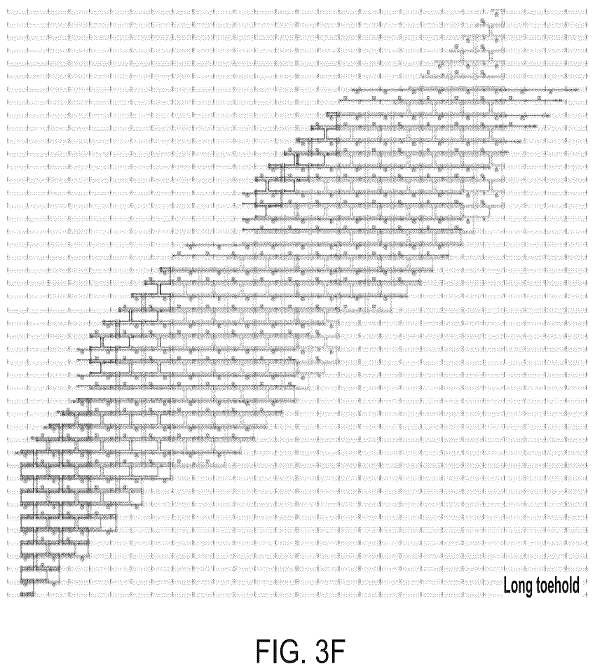

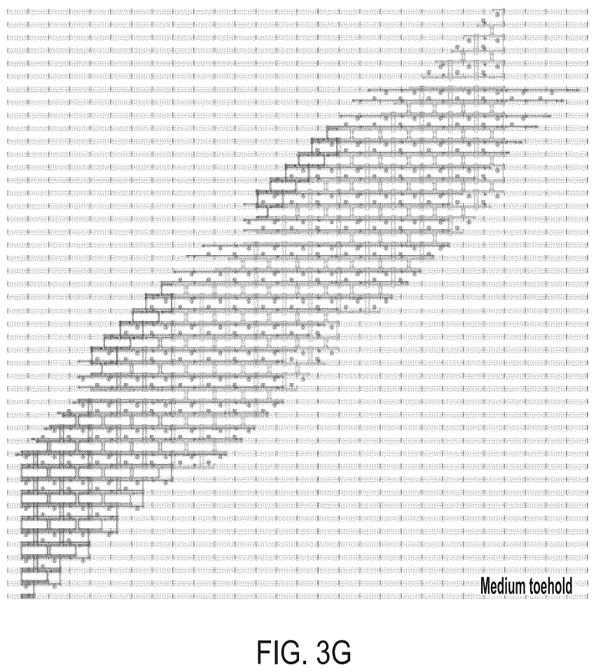

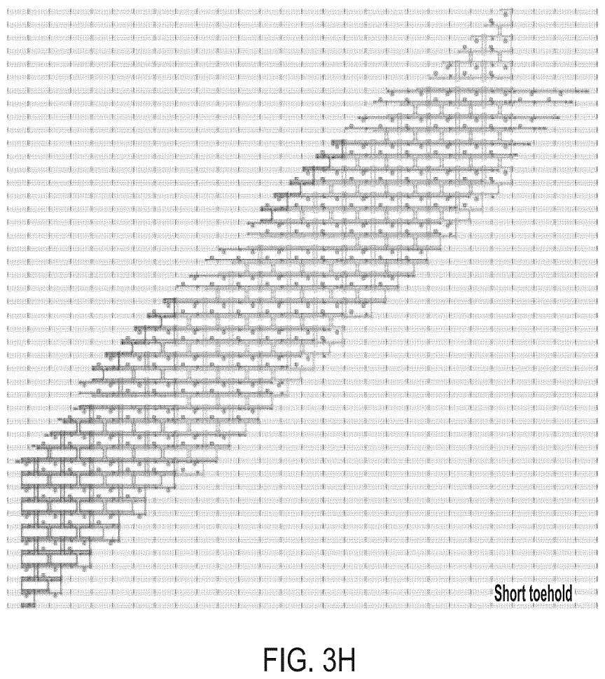

Following assembly, the crisscross ribbons were characterized using a 1% agarose gel prestained with SYBR Gold. As shown in FIG. 3A, the addition of the DNA origami seed resulted in formation of cut ribbon fragments.

### Example 3. The Length of a Toehold Domain can Vary the Rate of Amplification Via Ribbon Scission

Nucleic acid strands were purchased from Integrated DNA technologies (IDT), and were purified by denaturing polyacrylamide gel electrophoresis. Crisscross ribbons were assembled overnight at 52° C., using concentrations of 0.5 μM/growth-slat, 0.5 M/cut-slat, 16 mM MgCl₂, and 1 nM DNA origami seed. The growth-slats comprised a set of nucleic acid strands that bind to each other to form a crisscross ribbon comprising a first layer of strands and a second layer of strands, wherein the strands of the first layer comprise a single-stranded extension (which becomes a tochold domain) and were nonparallel to and complementary to the strands of the second layer. The cut-slats comprised slat strands, each of which was complementary to the growth-slats. The DNA origami seed comprised seed strands that were complementary to the growth-slats. In this Example, there were three sets of experimental conditions to vary the length of the tochold domain. In a first experiment, the single-stranded extension of the first layer of strands comprised 2 cooperative binding sites, each comprising 5-6 nucleotides (“short tocholds”). In a second experiment, the single-stranded extension of the first layer of strands comprised 3 cooperative binding sites, each comprising 5-6 nucleotides (“medium tocholds”). In a third experiment, the single-stranded extension of the first layer of strands comprised 4 cooperative binding sites, each comprising 5-6 nucleotides (“long tocholds”).

Following assembly, the crisscross ribbons were characterized using a 1% agarose gel prestained with SYBR Gold. As shown in FIG. 3B, the crisscross ribbon utilizing the long tocholds resulted in formation of the highest quantity of cut ribbon fragments. These data suggest that increasing the number of cooperative binding sites in a tochold domain increases the rate of cutting/scission.

### Example 4. Deletions or Wobble Basepairs can Increase the Rate of Amplification Via Ribbon Scission

Nucleic acid strands were purchased from Integrated DNA technologies (IDT) and were purified by denaturing polyacrylamide gel electrophoresis. Crisscross ribbons were assembled overnight at 52° C. using concentrations of 0.5 μM/growth-slat, 1 μM/cut-slat, 16 mM MgCl₂, and 1 nM DNA origami seed. The growth-slats comprised a set of nucleic acid strands that bind to each other to form a crisscross ribbon comprising a first layer of strands and a second layer of strands, wherein the strands of the first layer comprise a single-stranded extension (which becomes a tochold domain) and were nonparallel to and complementary to the strands of the second layer. The cut-slats comprised slat strands, each of which was complementary to the growth-slats. The DNA origami seed comprised seed strands that were complementary to the growth-slats. In a first experiment, the strands of the first layer were 100% complementary across a region to the strands of the second layer of the growth-slats. In a second experiment, the strands of the first layer were partially complementary across a region to the strands of the second layer of the growth-slats, with deletions present in the strands. In a third experiment, the strands of the first layer were partially complementary across a region to the strands of the second layer of the growth-slats, with wobble basepairs present between the strands.

Following assembly, the crisscross ribbons were characterized using a 1% agarose gel prestained with SYBR Gold. As shown in FIG. 3C, the use of crisscross ribbon having deletions or wobble basepairs between the strands resulted in formation of the highest quantity of cut ribbon fragments. These data suggest that inclusion of deletions or wobble basepairs between strands increases the rate of cutting/scission.

### Example 4. Amplification Via Ribbon Scission is Robust Across a Range of Temperatures

Nucleic acid strands were purchased from Integrated DNA technologies (IDT) and were purified by denaturing polyacrylamide gel electrophoresis. Crisscross ribbons were assembled overnight at variable temperature (46, 48, 50, or 52° C.), using concentrations of 0.5 μM/growth-slat, 0.5 μM/cut-slat, 16 mM MgCl₂, and 1 nM DNA origami seed. The growth-slats comprised a set of nucleic acid strands that bind to each other to form a crisscross ribbon comprising a first layer of strands and a second layer of strands, wherein the strands of the first layer comprise a single-stranded extension (which becomes a toehold domain) and were nonparallel to and complementary to the strands of the second layer. The cut-slats comprised slat strands, each of which was complementary to the growth-slats. The DNA origami seed comprised seed strands that were complementary to the growth-slats.

Following assembly, the crisscross ribbons were characterized using a 1% agarose gel prestained with SYBR Gold. As shown in FIG. 3D, the crisscross ribbon was assembled across the full range of tested temperatures. These data indicate that crisscross assembly methods described herein are robust, with no spurious nucleation detectable across a range of temperatures, and significantly more amplification is seen than in the linear growth condition.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising incubating in a reaction mixture:
(a) a set of nucleic acid strands that bind to each other to form a crisscross ribbon comprising a first layer of strands and a second layer of strands, wherein the strands of the first layer are nonparallel to and bound to the strands of the second layer,
(b) a biomolecule comprising seed strands to which strands of the first layer and/or second layer of the crisscross ribbon are bound; and
(c) slat strands, each of which binds to multiple strands of the crisscross ribbon that are bound to the seed strands, thereby displacing the seed strands from the crisscross ribbon.

2. The method of claim 1, wherein the biomolecule is a protein biomolecule, nucleic acid biomolecule, organic small molecule, or saccharide.

3. The method of claim 2, wherein the nucleic acid biomolecule is a DNA nanostructure.

4. A method, comprising incubating in a reaction mixture:
(a) a set of nucleic acid strands that bind to each other to form a crisscross ribbon comprising a first layer of strands and a second layer of strands, wherein the strands of the first layer are nonparallel to and bound to the strands of the second layer, and
(b) slat strands, each of which binds to multiple strands of the first layer of strands that are bound to the strands of the second layer, thereby displacing the strands of the first layer from the strands of the second layer.

5. The method of any one of the preceding claims, wherein the first layer of strands of the crisscross ribbon comprises parallel strands, and the second layer of strands of the crisscross ribbon comprises parallel strands that are perpendicular to and bound through cooperative binding sites to the strands of the first layer.

6. The method of claim 5, wherein each of the cooperative binding sites forms a helical half-turn.

7. The method of claim 5 or 6, wherein each of the cooperative binding sites comprises 3-10 nucleotide base pairs, optionally wherein each of the cooperative binding sites comprises 5-6 nucleotide base pairs.

8. The method of any one of the preceding claims, wherein the biomolecule comprises
(i) a first subset of seed strands to which a first subset of strands of the first layer of the crisscross ribbon are bound, and/or
(ii) a second subset of seed strands to which a second subset of strands of the second layer of the crisscross ribbon are bound.

9. The method of claim 8, wherein the slat strands comprise (i) a first subset of strands, each of which binds to multiple strands of the first layer of the crisscross ribbon and (ii) a second subset of strands, each of which binds to multiple strands of the second layer of the crisscross ribbon,
wherein binding of the slat strands to the strands of the crisscross ribbon displaces the crisscross ribbon from the seed strands, thereby displacing the crisscross ribbon from the biomolecule.

10. The method of any one of the preceding claims, wherein each nucleic acid strand of the first layer of strands comprises a single-stranded extension at one or both of its terminal ends.

11. The method of any one of the preceding claims, wherein each nucleic acid strand of the second layer of strands comprises a single-stranded extension at one or both of its terminal ends.

12. The method of any one of claims 5-11 comprising 2, 3, 4, 5, 6, 7, 8, 9 or 10 cooperative binding sites.

13. The method of any one of the preceding claims, wherein the nucleotides of the first layer of strands are complementary to the nucleotides of the second layer of strands.

14. The method of any one of the preceding claims, wherein the nucleotides of first layer of strands comprise at least one wobble base-pairing, mismatched base-pairing, or deletion relative to the nucleotides of second layer of strands that bind to the first layer.

15. The method of any one of the preceding claims, wherein displacing seed strands from the crisscross ribbon is performed by (i) toehold-mediated strand displacement; (ii) inclusion of gamma cut slats; inclusion of an engineered restriction site in a slat strand; (iii) inclusion of an unnatural or modified base in a strand of the crisscross ribbon and/or a slat strand; (iv) inclusion of small molecules that can function to accelerate scission in the reaction mixture; (v) inclusion of pH responsive elements in a strand of the crisscross ribbon and/or a slat strand; (vi) inclusion of a crosslinking or ligation junction in a strand of the crisscross ribbon and/or a slat strand; (vii) mechanical rupturing of crisscross ribbons; (viii) inclusion of photothermal elements in a strand of the crisscross ribbon and/or a slat strand; (ix) polymerase-based scission; (x) inclusion of thermoactivated slat strands; (xi) inclusion of nicking sites in a strand of the crisscross ribbon and/or a slat strand; and/or (xii) inclusion of exonucleases in the reaction mixture.

16. The method of any one of the preceding claims, wherein the method is performed at a temperature between 20-60° C., optionally 46-52° C.

17. A method of detection of a biomarker, comprising:
combining in a reaction mixture
(a) a set of nucleic acid strands that bind to each other to form a crisscross ribbon comprising a first layer of strands and a second layer of strands, wherein the strands of the first layer are nonparallel to and bound to the strands of the second layer,
(b) a biomolecule comprising seed strands to which strands of the first layer and/or second layer of the crisscross ribbon are bound, wherein the biomolecule seed is representative of the biomarker; and
(c) slat strands, each of which binds to multiple strands of the crisscross ribbon that are bound to the seed strands; and
incubating the reaction mixture under conditions that result in production of a branched nucleic acid nanostructure, wherein visualization of the nanostructure enables detection of the biomarker.

18. The method of claim 17, wherein the biomolecule comprises a biomarker binding partner that specifically binds to the biomarker.

19. A method of detection of a biomarker, comprising:
combining in a reaction mixture
(a) a sample comprising a biomarker; and
(b) a nucleic acid nanostructure comprising
(i) a nucleic acid scaffold strand and nucleic acid staple strands capable of assembling into multiple stacked parallel loops, and
(ii) a crisscross ribbon comprising a first layer of strands and a second layer of strands, wherein the strands of the first layer are nonparallel to and bound to the strands of the second layer, and slat strands, wherein each of the slat strands binds to multiple strands of the first layer of strands that are bound to the strands of the second layer, wherein the crisscross ribbon binds to the loops of (i);
wherein a biomarker binding partner that specifically binds to the biomarker is linked to each of the crisscross ribbons such that in the presence of the biomarker the biomarker binding partners bind to the biomarker and the nucleic acid nanostructure folds into multiple stacked parallel loops; and incubating the reaction mixture to assemble multiple stacked parallel loops.

20. The method of any one of claims 17-19, wherein the biomolecule seed is attached to the biomarker, optionally wherein the biomolecule seed is attached to the biomarker via an affinity agent.

21. The method of 20, wherein the biomolecule seed is a segment of the biomarker, wherein the biomarker comprises a nucleic acid.

22. The method of any one of claims 17-21, further comprising imaging the nanostructure.

23. The method of any one of claims 17-22, wherein each nucleic acid strand of the first layer of strands comprises a single-stranded extension at one or both of its terminal ends.

24. The method of any one of claims 17-23, wherein each nucleic acid strand of the second layer of strands comprises a single-stranded extension at one or both of its terminal ends.

25. The method of any one of claims 17-24, wherein the first layer of strands of the crisscross ribbon comprises parallel strands, and the second layer of strands of the crisscross ribbon comprises parallel strands that are perpendicular to and bound through cooperative binding sites to the strands of the first layer.

26. The method of claim 25, wherein each of the cooperative binding sites forms a helical half-turn.

27. The method of claim 26, wherein each of the cooperative binding sites comprises 3-10 nucleotide base pairs, optionally wherein each of the cooperative binding sites comprises 5-6 nucleotide base pairs.

28. The method of any one of claims 17-27, wherein the biomolecule comprises
(i) a first subset of seed strands to which a first subset of strands of the first layer of the crisscross ribbon are bound, and/or
(ii) a second subset of seed strands to which a second subset of strands of the second layer of the crisscross ribbon are bound.

29. The method of claim 24, wherein the slat strands comprise (i) a first subset of strands, each of which binds to multiple strands of the first layer of the crisscross ribbon and (ii) a second subset of strands, each of which binds to multiple strands of the second layer of the crisscross ribbon,
wherein binding of the slat strands to the strands of the crisscross ribbon displaces the crisscross ribbon from the seed strands, thereby displacing the crisscross ribbon from the biomolecule.

30. The method of any one of claims 17-29 comprising 2, 3, 4, 5, 6, 7, 8, 9 or 10 cooperative binding sites.

31. The method of any one of claims 17-30, wherein the nucleotides of the first layer of strands are complementary to the nucleotides of the second layer of strands.

32. The method of any one of claims 17-31, wherein the nucleotides of first layer of strands comprise at least one wobble base-pairing, mismatched base-pairing, or deletion relative to the nucleotides of second layer of strands that bind to the first layer.

33. The method of any one of claims 17-32, wherein the method is performed at a temperature between 20-60° C., optionally 46-52° C.

34. The method of any one of claims 17-33, wherein the biomarker binding partner that specifically binds to the biomarker is an antibody or aptamer.

35. A method of detection of an environmental change in a sample, comprising:
combining in the sample
(a) a set of nucleic acid strands that bind to each other to form a crisscross ribbon comprising a first layer of strands and a second layer of strands, wherein the strands of the first layer are nonparallel to and bound to the strands of the second layer, and
(b) slat strands, each of which binds to multiple strands of the first layer of strands that are bound to the strands of the second layer following an environmental change, thereby displacing the strands of the first layer from the strands of the second layer.

36. The method of claim 35, wherein the environmental change is a pH change, a temperature change, or a change in the concentration of one or more metal ions.
